(19) United States
(12) Patent Application Publication
(10) Pub. No.: US 2025/0257357 A1
(43) Pub. Date: Aug. 14, 2025

(54) TREATMENT OF H3.3-MUTANT BRAIN CANCER WITH PNKP INHIBITORS

(71) Applicants: UNIVERSITE PARIS CITE, Paris (FR); CENTRE NATIONAL DE LA RECHERCHE SCIENTIFIQUE (CNRS), Paris (FR); INSERM (INSTITUT NATIONAL DE LA SANTE ET DE LA RECHERCHE MEDICALE), Paris (FR)

(72) Inventors: Sophie POLO, Paris (FR); Béatrice RONDINELLI, Paris (FR); Giulia GIACOMINI, Paris (FR)

(21) Appl. No.: 18/856,827

(22) PCT Filed: Apr. 14, 2023
(86) PCT No.: PCT/EP2023/059855

§ 371 Date: Oct. 14, 2024

(30) Foreign Application Priority Data
Apr. 14, 2022 (EP) 22305555.9

(51) Int. Cl. C12N 15/113

(52) U.S. Cl. CPC C12N 15/1137; C12Y 301/03032; C12N 2310/14

(57) ABSTRACT

Despite aggressive radio/chemotherapy regimens, pediatric high-grade gliomas (pHGG) are deadly brain tumors that remain incurable and are the leading cause of cancer-related death in children. By analysing the impact of H3.3 mutations on DNA repair and genome integrity maintenance capacities of glioma cells, the present inventors identified the PNKP enzyme as being a major protein partner interacting with mutated H3 oncohistone specifically, and involved in DNA aberrant repair. They showed that inhibition of this enzyme prevents the proliferation of glioma tumor cells bearing specific H3 oncohistone mutations. They therefore propose to target this enzyme in order to efficiently treat patients suffering from gliomas, in particular pediatric gliomas bearing these specific H3 oncohistone mutations, or to sensitize them to current radio/chemotherapeutic regimens, for which there is very limited response.

### SUMMARY OF THE INVENTION

Despite aggressive radio/chemotherapy regimens, pediatric high-grade gliomas (pHGG) are deadly brain tumors that remain incurable and are the leading cause of cancer-related death in children. By analysing the impact of H3.3 mutations on DNA repair and genome integrity maintenance capacities of glioma cells, the present inventors identified the PNKP enzyme as being a major protein partner interacting with mutated H3 oncohistone specifically, and involved in DNA aberrant repair. They showed that inhibition of this enzyme prevents the proliferation of glioma tumor cells bearing specific H3 oncohistone mutations. They therefore propose to target this enzyme in order to efficiently treat patients suffering from gliomas, in particular pediatric gliomas bearing these specific H3 oncohistone mutations, or to sensitize them to current radio/chemotherapeutic regimens, for which there is very limited response.

### BACKGROUND OF THE INVENTION

Pediatric high-grade gliomas (pHGG) are deadly brain tumors characterized by less than 10% survival rate within 2 years of diagnosis. Despite aggressive radio/chemotherapy regimens, pHGG remain incurable and are the leading cause of cancer-related death in children. The toxicity of existing therapies and the emergence of resistance hinder treatment and call for the development of alternative, targeted therapeutic strategies exploiting specific molecular features of cancer cells. Among them, heterozygous, dominant point mutations in the H3F3A gene encoding the H3.3 histone variant are recurrent in pHGG and active players in the oncogenic process (Weinberg D N et al, 2017). These mutually exclusive mutations result in single amino-acid substitutions at Lysine 27 to Methionine (K27M), giving rise to midline tumors, and at Glycine 34 to Arginine or Valine (G34R/V) in cerebrocortical tumors (Weinberg D N et al, 2017). These mutations perturb histone post-translational modifications (PTMs) in cis or in trans (Weinberg D N et al, 2017) thus interfering with genome-wide gene expression programs that stall the differentiation of interneuron progenitor cells at different stages and facilitate transformation (reviewed in Deshmukh S. et al, 2021).

Besides their effect on gene expression, pHGG H3.3 mutations also promote genome instability (Pfister, S. X. et al. 2014; Yadav, R. K. et al. 2017; Day, C. et al, 2021) known as a major driver of cellular transformation. In particular, H3.3 mutant pHGGs display increased Copy Number Alterations (CNA) and chromosomal rearrangements compared to wild-type H3.3 tumors (Ferrand J. et al, 2020). Mechanistically, works in various model systems show that G34 mutations in fission yeast and the non-pHGG H3.3K36M mutation in human cells hijack the response to DNA damage by interfering in a dominant manner with the DNA repair-promoting function of wild-type H3.3 (Pfister, S. X. et al. 2014; Yadav, R. K. et al. 2017). Moreover, G34 mutations alter H3.3 association with several DNA repair factors in human cells (Lim, J. et al 2018). This expands the role of mutated H3.3 histone proteins beyond their ability to dysregulate gene expression and paves the way for the identification of additional oncogenic functions. The impact of pHGG H3.3 oncohistone mutations on the DNA repair and genome integrity maintenance capacities of human cells, whether and how this function facilitates transformation and can be exploited for therapy, remains however unknown.

In this context, the present inventors have analysed more deeply how H3 mutations impact DNA repair, in order to identify key factors that could serve as efficient therapeutic targets for controlling the proliferation of tumor cells bearing H3 oncohistone mutations.

### DESCRIPTION OF THE INVENTION

By dissecting how H3.3 mutants skew DNA repair, the present inventors herein show that mutations in core chromatin components induce genome instability by dysregulating the recruitment of repair factors at damaged replication forks (RFs) and by conferring a gain-of-function to histone H3.3 independent from hypomethylation of H3 K27 and K36. Their data establish for the first time that the deposition of H3.3K27M and G34R mutants at damaged RFs is associated with dysregulated histone PTMs that may in turn affect fork repair by impacting on the recruitment of repair factors.

In this context, the DNA end processing enzyme called “Polynucleotide Kinase 3′-Phosphatase” (PNKP) is identified for the first time to be specifically associated with both K27M and G34R, and not with wild-type H3.3 histone protein. The PNKP enzyme, which contributes to NHEJ by transferring a phosphate group between broken DNA ends before ligation (Dumitrache, L. C. & Mckinnon, P. J, 2017), plays a central role in neurodevelopment (Dumitrache, L. C. & Mckinnon, P. J, 2017). Importantly, increased binding of PNKP in H3.3K27M and G34R cells was observed by two different experimental means, namely by proximity-dependent biotinylation (BioID) and by isolation of proteins on nascent DNA (iPOND). These results substantiate the preferential association of this particular DNA repair enzyme with both H3.3 mutants. Moreover, the results of the inventors show for the first time that the PNKP association with H3.3K27M and G34R drives aberrant NHEJ in cells expressing these mutants. As a matter of fact, PNKP siRNA knockdown reduces NHEJ in H3.3K27M and G34R cells, but not in wild-type H3.3 cells (FIG. 4C). Finally, the results of the inventors show that PNKP knockdown specifically impairs the growth of H3.3K27M and G34R U2OS cells and of glioma cells harboring endogenous H3.3 G34R or K27M mutations (HSJD cells and MGBM1 cells or SU-DIPG-XVII cells respectively), but not the growth of wild-type or G34V mutant H3.3 cells, which remains unaffected (FIG. 4E). Importantly, these data demonstrate that PNKP is a potential therapeutic target in pHGG cells expressing specific H3.3 mutations.

Targeting DNA repair defects is the most developed approach so far to induce tumor cell killing through synthetic lethality, even if weakly exploited beyond BRCA-mutated tumors. By providing molecular understanding of the aberrant DNA repair capacities of H3.3 mutant pHGGs, it is herein proposed the first, synthetic lethal approach for the treatment of these tumors by targeting and inhibiting PNKP enzyme. PNKP functions both in NHEJ and SSBR repair pathways (Dumitrache, L. C. & Mckinnon, P. J, 2017) and both could support the synthetic lethality observed. PNKP inhibition has been proposed to kill PTEN-deficient cancers (Mereniuk, T. R. et al. 2013) and could also be exploited to sensitize glioma cells to current radio/chemotherapeutic regimens, for which there is very limited response. Moreover, the specificity towards H3.3K27M and G34R mutant cells should limit toxicity-related side effects in future treatments and opens up the possibility to employ a similar strategy in other cancers bearing the same H3.3 mutations.

In a first aspect, the present invention relates to the use, in particular the in vivo, ex vivo or in vitro use, of an inhibitor of the bifunctional polynucleotide kinase/phosphatase (PNKP) enzyme for preparing a medicament that can inhibit or prevent the proliferation of tumor cells bearing at least one H3 oncohistone mutation. In other terms, the invention relates to an inhibitor of the bifunctional polynucleotide kinase/phosphatase (PNKP) enzyme for use for inhibiting or preventing the proliferation of tumor cells bearing at least one H3 oncohistone mutation, in particular in a subject. Also in other terms, the invention relates to a method for inhibiting or preventing the proliferation of tumor cells bearing at least one H3 oncohistone mutation in a subject in need thereof, comprising administering to the subject an effective amount of an inhibitor of the bifunctional polynucleotide kinase/phosphatase (PNKP) enzyme.

Preferably, the tumor to be treated contains tumor cells carrying a H3 oncohistone mutation that induces an increased binding of PNKP to the mutated histone or to damaged replication forks in said tumor cells. Preferably, these tumors are neither lung cancer, nor prostate cancer, nor colon cancer.

The increased binding of PNKP to the mutated histone can be detected by any direct experimental means, such as, in particular, proteomic analysis, proximity-dependent biotinylation (BioID) or by isolation of proteins on nascent DNA (iPOND). It is also possible to detect this binding indirectly, for example by assessing the number of S-phase specific RAD51 and 53BP1 foci in the nuclei of the tumor cells by immunofluorescence. Increased binding of PNKP to mutated histone or to damaged replication forks would indeed result to an enhanced number of RAD51 and 53BP1 foci in the nuclei of the tumor cells, e.g., an enhancement of at least 50%, preferably of at least 80%, of the number of said nuclear foci as compared with control undamaged cells or to WT cells. This detection can be done by immunofluorescent staining of repair foci (see also in the experimental part below).

The inhibitor of the invention enables to treat cancer by impairing or limiting the proliferation of tumor cells in which the H3 histone proteins carry a somatic dominant mutation that contributes to drive tumorigenesis (also called a “H3 oncohistone mutation”). This mutation can be a missense mutation modifying histone PTMs, such as those disclosed in Weinberg D N et al, 2017 and in Ferrand J. et al, 2020.

Human cells have eleven known sequence variants of histone H3. These are denoted as Histone H3.1, Histone H3.2, Histone H3.3, Histone H3.4 (H3T), Histone H3.5, Histone H3.6, Histone H3.7, Histone H3.8, Histone H3.Y.1 (H3.Y), Histone H3.Y.2 (H3.X), and the centromeric variant CENP-A. They have highly conserved sequences, which for some of them differ only by a few amino acids.

Variant H3.3 was found to play an important role in maintaining genome integrity during mammalian development and a number of oncohistone mutations have been identified in said variant, notably in paediatric glioblastoma (Weinberg D N et al, 2017; Ferrand J. et al, 2020). Therefore, in a particular embodiment, the cancer/tumor cells treated with the inhibitor of the invention carry a H3 oncohistone mutation that affects histone variant H3.3.

A number of oncogenic mutations have been identified in said variant H3.3. They are for example K27M, G34R, G34V, G34W, and K36M (Weinberg D N et al, 2017). In a preferred embodiment, the cancer/tumor cells treated with the inhibitor of the invention bear at least one mutation in the H3.3 histone variant, said mutation being chosen in the group consisting of: G34W, K36M, K27M and G34R.

In one particular embodiment, the inhibitor of the invention is used to treat glioma tumors, such as paediatric glioma, whose cells bear the mutation K27M or G34R on histone variant H3.3.

In one particular embodiment, the inhibitor of the invention is used to treat giant cell tumor of bone bearing the mutation G34W on histone variant H3.3.

In one particular embodiment, the inhibitor of the invention is used to treat chondroblastoma cells bearing the mutation K36M on histone variant H3.3.

Variant H3.1 also harbors the K27M mutation in glioblastoma (Weinberg D N et al, 2017; Ferrand J. et al, 2020), that was shown to affect DNA repair (Weinberg D N et al, 2017; Ferrand J. et al, 2020). Therefore, in a particular embodiment, the tumor/cancer cells treated with the inhibitor of the invention carry a H3 oncohistone mutation that affects histone variant H3.1, such as H3.1K27M.

Thus, the inhibitor of the invention can be used to prevent or limit the proliferation of tumors that are chosen in the group consisting of: glioma, osteosarcoma, adrenocortical carcinoma, giant cell tumor of bone, chondroblastoma and acute myeloid leukemia (AML), provided that these cells carry a H3 oncohistone mutation (preferably on the H3.3 variant) that induces an increased binding of PNKP to mutated histone or to damaged replication forks in said tumor cells, as explained above.

In a particularly preferred embodiment, the inhibitor of the invention is used to treat glioma such as paediatric glioma bearing at least one H3.3 oncohistone mutation showing an increased binding of PNKP to mutated histone or to damaged replication forks in said tumor cells.

Preferably, the inhibitor of the invention is administered to tumor cells (in particular glioma cells) that have unmutated PTEN, ING3, CDKN3, PTPN6 and/or SMG1 genes and/or normal expression of any of the following proteins: PTEN, ING3, CDKN3, PTPN6 and/or SMG1.

The inhibitor of the invention can be of any nature, provided that it is able to inhibit the expression or the function of the bifunctional polynucleotide kinase/phosphatase (PNKP) enzyme. This enzyme is also called “Polynucleotide Kinase 3′-Phosphatase” or “DNA 5′ kinase/3′ phosphatase”. In humans, it has the sequence of SEQ ID NO:2. It includes two enzymatic domains: a polynucleotide 3′ phosphatase (EC: 3.1.3.32) and a polynucleotide 5′ hydroxyl-kinase (EC 2.7.1.78). It plays a key role in the repair of DNA damage, functioning as part of both the non-homologous end-joining (NHEJ) and base excision repair (BER) pathways. Through its two catalytic activities (kinase/phosphatase), PNKP ensures that DNA termini are compatible with extension and ligation by removing 3′-phosphates and/or by phosphorylating 5′-hydroxyl groups on the ribose sugar of the DNA backbone (Jilani A et al, 1999, Karimi-Busheri F et al, 1999). As intended herein, inhibiting the function of PNKP includes inhibiting the interaction or the binding of PNKP with/to one or more of its cellular targets or partners, such as PNKP-binding proteins.

In particular, said PNKP inhibitor can be a small chemical drug, a peptide, an antibody or an aptamer that prevents PNKP enzyme from binding on DNA breaks, that inhibits the binding of PNKP to a PNKP-binding protein, or that inhibits its kinase activity or its phosphatase activity. Consequently, PNKP normal biological activity is prevented or reduced. The antagonistic activity of these inhibitors towards the PNKP enzyme may be determined through the TAMRA assay (Kalasova et al., 2019). For example, the agents may be tested for their capacity to remove 3′-phosphates from, or to phosphorylate 5′-hydroxyl groups on, the ribose sugar of the DNA backbone.

In one embodiment, the inhibitor of the invention is a small chemical drug such as A12B4C3 (CAS number 1005129-80-5) or its derivative A83B4C63, which are well-known non-toxic PNKP inhibitors (Freschauf G K et al, 2009).

In another embodiment, the inhibitor of the invention consists in an antibody (the term including antibody fragment). In particular, the inhibitor may consist in an antibody directed against the PNKP enzyme in such a way that said antibody impairs the removing of 3′-phosphates from, or the phosphorylation of 5′-hydroxyl groups on the ribose sugar of the DNA backbone. Antibodies can be raised according to known methods by administering the appropriate antigen (here PNKP) or epitope to a host animal selected, e.g., from pigs, cows, horses, rabbits, goats, sheep, and mice, among others. Various adjuvants known in the art can be used to enhance antibody production. Although antibodies useful in practicing the invention can be polyclonal, monoclonal antibodies are preferred. Monoclonal antibodies can be prepared and isolated using any technique that provides for the production of antibody molecules by continuous cell lines in culture. Techniques for production and isolation include but are not limited to the hybridoma technique; the human B-cell hybridoma technique; and the EBV-hybridoma technique. Alternatively, techniques described for the production of single chain antibodies (see, e.g., U.S. Pat. No. 4,946,778) can be adapted to produce anti-PNKP, single chain antibodies.

The inhibitor of the invention also includes anti-PNKP antibody fragments including but not limited to F(ab′)₂ fragments, which can be generated by pepsin digestion of an intact antibody molecule, and Fab fragments, which can be generated by reducing the disulfide bridges of the F(ab′)₂ fragments. Alternatively, Fab and/or scFv expression libraries can be constructed to allow rapid identification of fragments having the desired specificity to the receptor or channel.

Humanized antibodies and antibody fragments therefrom can also be prepared according to known techniques. “Humanized antibodies” are forms of non-human (e.g., rodent) chimeric antibodies that contain minimal sequence derived from non-human immunoglobulin. For the most part, humanized antibodies are human immunoglobulins (recipient antibody) in which residues from a hypervariable region (CDRs) of the recipient are replaced by residues from a hypervariable region of a non-human species (donor antibody) such as mouse, rat, rabbit or nonhuman primate having the desired specificity, affinity and capacity. In some instances, framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence.

The humanized antibody optionally also will comprise at least a portion of an immunoglobulin constant region (Fc), typically that of a human immunoglobulin. Methods for making humanized antibodies are described in the art. After producing antibodies as above described, the skilled man in the art can easily select those blocking the PNKP enzyme. Anti-PNKP antibodies, antibody fragments and single-chain antibodies can also be produced in situ, i.e. within cells, using nucleic acids encoding them, such as mRNA vaccines.

In another embodiment, the inhibitor of the PNKP enzyme activity of the invention is an aptamer. Aptamers are a class of molecule that represents an alternative to antibodies in term of molecular recognition. Aptamers are oligonucleotide or oligopeptide sequences with the capacity to recognize virtually any class of target molecules with high affinity and specificity. Such ligands may be isolated through Systematic Evolution of Ligands by Exponential enrichment (SELEX) of a random sequence library. The random sequence library is obtainable by combinatorial chemical synthesis of DNA. In this library, each member is a linear oligomer, eventually chemically modified, of a unique sequence. Peptide aptamers consists of a conformationally constrained antibody variable region displayed by a platform protein, such as E. coli Thioredoxin A that are selected from combinatorial libraries by two hybrid methods. After raising aptamers directed against the PNKP enzyme as above described, the skilled man in the art can easily select those blocking the PNKP enzyme.

The inhibitor of the invention can also prevent or reduce the expression of the PNKP mRNA-encoded protein. As used herein, an “inhibitor of mRNA expression” refers to a natural or synthetic compound that has a biological effect to inhibit or significantly reduce the expression of a mRNA. Inhibitors of mRNA expression for use in the present invention are for example anti-sense oligonucleotide constructs. Anti-sense oligonucleotides, including anti-sense RNA molecules and anti-sense DNA molecules, act to directly block the translation of the mRNA by binding thereto and thus preventing protein translation or increasing mRNA degradation, thus decreasing the level of the protein (e.g. the PNKP protein), and thus its activity, in the target cell. Antisense oligonucleotides of at least about 15 bases and complementary to unique regions of the mRNA transcript sequence encoding the targeted PNKP protein (e.g., SEQ ID NO:1) can be used. Said oligonucleotide constructs can be synthesized, e.g., by conventional phosphodiester techniques, and administered to the patients, by e.g., intravenous injection or infusion. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732).

In particular, small inhibitory RNAs (siRNAs) or microRNAs (miRNAs) can function as inhibitors of the PNKP mRNA expression for use in the present invention. PNKP mRNA expression can be reduced by contacting a subject or cell with a small double stranded RNA (dsRNA), or a vector or construct causing the production of a small double stranded RNA, such that PNKP mRNA expression is specifically inhibited (i.e. by RNA interference or RNAi). Methods for selecting an appropriate dsRNA or dsRNA-encoding vector are well known in the art, in particular for the genes whose sequence is known. For example, siRNAs affecting PNKP expression are proposed in the art (Kalasova et al, 2019). Short hairpin RNAs (shRNAs) and/or locked nucleic acids (LNA) can also be used, as disclosed in Mereniuk T R et al, 2012.

The PNKP enzyme is encoded in humans by the PNKP gene located on the Chr19 q13.33, whose mRNA has the sequence NM_007254 (SEQ ID NO:1). The encoded enzyme has the polypeptide sequence NP_009185.2 (SEQ ID NO:2).

In a particularly preferred embodiment, the inhibitor of the invention is an anti-sense oligonucleotide that impairs, reduces or prevents the expression of the PNKP mRNA-encoded protein. In a more particular embodiment, the inhibitor of the invention is a siRNA or a shRNA that impairs, reduces or prevents the formation of the mRNA of SEQ ID NO:1.

The decreased expression level of the PNKP mRNA in tumor cells can be assessed by any conventional mean, e.g. by RT-PCR using dedicate oligonucleotide primers or indirectly by evaluating the downregulation of the protein by western blot on total cell extracts using PNKP specific antibodies. The skilled person well knows the conditions and the settings of RT-PCR assays that should be used to detect the expression level of the PNKP gene in tumor cells.

In a particular embodiment, the inhibitor of the invention is a siRNA whose sequence is:

siPNKP_3′UTR_1:
(SEQ ID NO: 3)
5′-CAG CUC CCC UCC ACA AUA A-3′
siPNKP_3′UTR_2:
(SEQ ID NO: 4)
5′-CCU CCA CAA UAA ACG CUG U-3′
siPNKP_3′UTR_3:
(SEQ ID NO: 26)
5′-CCA CAA UAA ACG CUG UUU C-3′.

Ribozymes can also function as inhibitors of gene expression for use in the present invention. Ribozymes are enzymatic RNA molecules capable of catalysing the specific cleavage of RNA. The mechanism of ribozyme action involves sequence specific hybridization of the ribozyme molecule to complementary target RNA, followed by endonucleolytic cleavage. Engineered hairpin or hammerhead motif ribozyme molecules that specifically and efficiently catalyse endonucleolytic cleavage of mRNA sequences are thereby useful within the scope of the present invention. Specific ribozyme cleavage sites within any potential RNA target are initially identified by scanning the target molecule for ribozyme cleavage sites, which typically include the following sequences, GUA, GUU, and GUC. Once identified, short RNA sequences of between about 15 and 20 ribonucleotides corresponding to the region of the target gene containing the cleavage site can be evaluated for predicted structural features, such as secondary structure, that can render the oligonucleotide sequence unsuitable. The suitability of candidate targets can also be evaluated by testing their accessibility to hybridization with complementary oligonucleotides, using, e.g., ribonuclease protection assays.

Both antisense oligonucleotides and ribozymes useful as inhibitors of gene expression can be prepared by known methods. These include techniques for chemical synthesis such as, e.g., by solid phase phosphoramadite chemical synthesis. Alternatively, anti-sense RNA molecules can be generated by in vitro or in vivo transcription of DNA sequences encoding the RNA molecule. Such DNA sequences can be incorporated into a wide variety of vectors that incorporate suitable RNA polymerase promoters such as the T7 or SP6 polymerase promoters. Various modifications to the oligonucleotides of the invention can be introduced as a means of increasing intracellular stability and half-life. Possible modifications include but are not limited to the addition of flanking sequences of ribonucleotides or deoxyribonucleotides to the 5′ and/or 3′ ends of the molecule, or the use of phosphorothioate or 2′-O-methyl rather than phosphodiesterase linkages within the oligonucleotide backbone.

Antisense oligonucleotides, siRNAs and ribozymes of the invention may be delivered into tumors in association with a vector. In its broadest sense, a “vector” is any vehicle capable of facilitating the transfer of the antisense oligonucleotide siRNA or ribozyme nucleic acid to the tumor cells bearing at least one H3 oncohistone mutation. Preferably, the vector transports the nucleic acid to the tumor cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide siRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rouse sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

In a particular embodiment, the inhibitor of the invention is combined with an effective carrier or drug delivery system, for targeting in particular glioblastoma cells that are located beyond the blood-brain barrier and therefore difficult to reach. It is for example coupled to complex nanocarriers such as magnetic nanoparticles, nanotubes, liposomes, polymeric or inorganic nanoparticles, microvesicles, implants or micelles. These carriers can be designed by several approaches using lipidic and/or polymeric materials. The selection of the most suitable material depends on the physiochemical characteristics and interactions between the system and the molecule to be integrated, the route of administration (Moras et al, 2021).

In a particularly preferred embodiment, the inhibitors of the invention are siRNAs (e.g., of SEQ ID NO: 3, 4 or 26) that are encapsulated in liposomes so as to protect them from environmental degradation.

These various systems carrying nucleic acid inhibitors or drug compounds can be administered by any convenient route. The administration can be systemic, preferentially intravenously, oral or parenteral. In a preferred embodiment, the administration is local, e.g., intra-tumorally, in order to be uptaken in vivo by the patient's tumor cells and bypass the blood-brain barrier. Different ways to enhance the uptake of drugs in brain cells are disclosed in Moras et al, 2021. It is for example possible to use a local implant, a convection-enhanced delivery, a direct intratumoral administration or to disrupt the blood-brain barrier by opening the tight junctions for a short period and in a specific site. The administration can then be done by intravascular delivery, intracerebral delivery, intranasal delivery, intracarotid infusion, interstitial delivery, transmucosal delivery, etc (Moras et al, 2021).

In a particular embodiment, the inhibitors of the invention, or the vectors carrying same, are included within a pharmaceutical composition also containing excipients or vehicles which are pharmaceutically acceptable. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions. The pharmaceutical forms suitable for injectable use include sterile aqueous solutions or dispersions; formulations including sesame oil, peanut oil or aqueous propylene glycol; and sterile powders for the extemporaneous preparation of sterile injectable solutions or dispersions. In all cases, the form must be sterile and must be fluid to the extent that easy syringability exists. It must be stable under the conditions of manufacture and storage and must be preserved against the contaminating action of microorganisms, such as bacteria and fungi. Solutions comprising compounds of the invention as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations contain a preservative to prevent the growth of microorganisms. The active ingredient can be formulated into a composition in a neutral or salt form. Pharmaceutically acceptable salts include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. Sterile injectable solutions are prepared by incorporating the active agent in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. Upon formulation, solutions will be administered in a manner compatible with the dosage formulation and in such amount as is therapeutically effective. The formulations of the invention are easily administered in a variety of dosage forms, such as the type of injectable solutions described above, but drug release capsules and the like can also be employed. For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous and intraperitoneal administration.

The present inventors show in the experimental part below that inhibition of the PNPK enzyme (e.g., by siRNA means) prevents the proliferation of glioma tumor cells bearing specific H3 oncohistone mutations. The present inventors therefore propose efficient methods to treat patients suffering from gliomas, in particular pediatric gliomas, or to sensitize them to current radio/chemotherapeutic regimens. These methods involve the administration of a therapeutically effective amount of the inhibitors of the invention, or of a vector carrying same, or of a pharmaceutical composition containing same, optionally along with a radio- or chemotherapeutic treatment.

As used herein, the terms “treat”, “treating”, “treatment”, and the like refer to reducing or ameliorating the symptoms of a disorder (e.g., pediatric glioma), and/or symptoms associated therewith. It will be appreciated that, although not precluded, treating a disorder or condition does not require that the disorder, condition or symptoms associated therewith be completely eliminated.

In a further aspect, the present invention relates to the use of the above-mentioned pharmaceutical compositions in methods for treating subjects in need thereof.

As used herein, the term “subject” relates to a mammalian, in particular a human, subject that will benefit or that is likely to benefit from the methods and pharmaceutical compositions of the present invention. The subject may suffer from a tumor carrying a H3 oncohistone mutation, such as glioma, osteosarcoma, adrenocortical carcinoma, giant cell tumor of bone, chondroblastoma and acute myeloid leukemia (AML), as disclosed above. These cancers can be of any stage such that it could be an early non-invasive cancer or could be a late stage cancer that has already progressed to form metastases in the body. In a particular embodiment, this tumor has unmutated PTEN, ING3, CDKN3, PTPN6 and/or SMG1 genes and/or normal expression of any of the following proteins: PTEN, ING3, CDKN3, PTPN6 and/or SMG1. In another particular embodiment, this tumor is not a lung, breast, prostate or colon cancer.

By “therapeutically effective amount” is herein meant a sufficient amount of the inhibitor at a reasonable benefit/risk ratio applicable to the medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the subject; the time of administration, route of administration, and rate of excretion of the specific compound employed; the duration of the treatment; drugs used in combination or coincidental with the specific polypeptide employed; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per Kg body weight, per day. Preferably, the compositions contain 0.01, 0.05, 0.1, 0.5, 1.0, 2.5, 5.0, 10.0, 15.0, 25.0, 50.0, 100, 250 and 500 mg of the inhibitor for the symptomatic adjustment of the dosage to the subject to be treated.

In a particular embodiment, the inhibitor of the invention, or the pharmaceutical composition comprising same, is administered to the subject in need thereof in combination with a radiotherapeutic treatment. As a matter of fact, the compositions of the invention are likely to enhance the efficiency of a radiotherapy treatment.

In other words, the present invention targets the use of the inhibitors mentioned above for manufacturing a pharmaceutical composition intended to be administered prior or after radiotherapy to a subject in need thereof. It is also drawn to the use of the inhibitors mentioned above for manufacturing a pharmaceutical composition intended to potentiate a radiotherapy treatment to a subject in need thereof. It is finally drawn to the use of the inhibitors mentioned above for manufacturing a pharmaceutical composition intended to treat cancer, in conjunction with radiotherapy, in a subject in need thereof.

Exemplary radiotherapeutic protocols are well-known in the art. They are for example from 40 to 60 Grays (Gy) for glioma tumors, in one or several administrations. Many other factors are considered by radiation oncologists when selecting a dose, including whether the patient is also receiving chemotherapy, patient comorbidities, whether radiation therapy is being administered before or after surgery, and the degree of success of surgery.

In one embodiment, administering the compositions of the invention will enable to reduce the dose of irradiation and therefore results in the alleviation of the side effects incurred by the radiation treatment.

In an embodiment, the compositions of the present invention are administered before or after the radiotherapy treatment, typically 24 h before or after radiotherapy is applied. The time of administration will depend on the specific formulation and on the time necessary for the inhibitors to reach the target tumor cells. The time of administration will be chosen so as to provide the optimal concentration of the inhibitors or of the cell compositions of the invention, at the time of irradiation.

In another embodiment, the administration of the compositions of the present invention and the radiotherapy treatment are performed concomitantly.

The term “irradiation therapy” is commonly used in the art to refer to multiple types of radiation therapy including internal and external radiation therapy, radio-immunotherapy, and the use of various types of radiation including X-rays, gamma rays, alpha particles, beta particles, photons, electrons, neutrons, radioisotopes, and other forms of ionizing radiation. As used herein, the terms “irradiation therapy”, “radiation therapy”, “radiation” and “irradiation” are inclusive of all of these types of radiation therapy, unless otherwise specified. There are different types of radiotherapy machines, which work in slightly different ways. The number and duration of the radiotherapy sessions depend on the type of cancer and where it is located in the body. A superficial skin cancer may need only a few short treatments, whereas a cancer deeper in the body may need more prolonged treatment.

In a particular embodiment, the inhibitor of the invention, or the pharmaceutical composition comprising same, is administered to the subject in need thereof in combination with a chemotherapeutic treatment such as temozolomide or camptothecin, or any other efficient treatment that impairs pHGG development.

The invention will be further illustrated by the following figures and examples. However, these examples and figures should not be interpreted in any way as limiting the scope of the present invention.

### FIGURE LEGENDS

FIG. 1 shows how K27MH3.3 and G34R pHGG mutations hijack DNA repair in S phase and harbor genome instability features of aberrant NHEJ. (a-b), Analysis of RAD51 (a) and 53BP1 (b) repair foci by immunofluorescence in U2OS cells stably expressing wildtype H3.3 (WT_1) or the indicated mutants and treated with camptothecin (3 h, 0.1 μM). Representative images of repair foci in EdU⁺ cells are shown. Bar graphs depict the percentage of EdU⁺ and EdU⁻ cells harboring more than 5 (RAD51) or 10 (53BP1) foci. Mean±s. e. m from three independent experiments, with n>117 for each experiment. (c), Analysis of NHEJ activity by Random Plasmid Integration Assay (RPIA) in U2OS cells stably expressing wildtype (WT_1) or mutant H3.3. Vector-, negative untransfected control. (d), Serial dilution analyses and proliferation curves of S. pombe strains expressing wildtype or mutant H3, depleted for the core NHEJ factor xrc4 (Axrc4) and grown in standard growth medium (YEA) or in the presence of camptothecin. (e), Scoring of radials in metaphase spreads of U2OS cells stably expressing wildtype (WT) or mutant H3.3 and treated with Mitomycin C (24 hs, 25 ng/ml). Cells transfected with siRNA against FANCD2 (siFANCD2) are used as positive control (siLUC: siLuciferase, control). A representative example of a radial chromosome is shown in the inset. The western blot shows siRNA efficiency (Tubulin, loading control). Mean±s. e. m from two independent experiments, with n>30 per sample for each experiment. (f), Analysis of DNA repair-driven mutational signatures (SBS3, single-base substitutions 3; ID8, indels 8) in whole-genome sequences of pre-treatment, TP53-mutated, primary pHGG samples harboring wildtype (WT), K27M or G34R H3.3. Statistical significance is calculated by two-way ANOVA (a, b), one-way Anova with Bonferroni post-test (c, e) or the non-parametric Kruskal-Wallis test (f). *: p<0.05; **: p<0.01; ***: p<0.001; ns: p>0.05. Scale bars, 10 μm.

FIG. 2 shows that H3.3 pHGG mutants hinder DNA repair through a gain-of-function mechanism independently of hypomethylation at H3 lysines 27 and 36. (a), Analysis of RAD51 and 53BP1 foci by immunofluorescence in U2OS cells transfected with siRNAs against Luciferase (siLUC, control) or H3.3 (siH3.3) and treated with camptothecin (3 h, 0.1 μM). The western blot shows siRNA efficiency (Tubulin, loading control). Representative images of RAD51 and 53BP1 foci in EdU⁺ cells are shown. Bar graphs depict the percentage of EdU⁺ and EdU⁻ cells harboring more than 5 (RAD51) or 10 (53BP1) foci. Mean±s. e. m from two independent experiments, with n>113 for each experiment. (b), Analysis of RAD51 and 53BP1 foci by immunofluorescence in Hela cells treated with DMSO or the EZH2 inhibitor GSK126 (EZH2i, 72 h, 1 μM) and damaged with camptothecin (3 h, 0.1 μM). The western blot shows the efficiency of EZH2 inhibition by analysing levels of H3K27me3. Bar graphs depict the number of RAD51 or 53BP1 foci per cell in EdU⁺ and EdU⁻ cell populations. Mean±s. e. m from three independent experiments, with n>125 for each experiment. (c), Analysis of RAD51 and 53BP1 foci by immunofluorescence in U2OS cells transfected with siRNAs against Luciferase (siLUC, control), RNF168 (siRNF168) or SETD2 (siSETD2) and treated with camptothecin (3 h, 0.1 μM). siRNA efficiencies and H3K36me3 levels are analyzed by western blot (Tubulin, loading control). Bar graphs depict the number of RAD51 or 53BP1 foci per cell in each condition. As a control, RNF168 protein, that was shown to abolish both RAD51 and 53BP1 foci formation, was depleted. Mean±s. e. m from three independent experiments, with n>131 for each experiment. Statistical significance is calculated by two-way ANOVA with Dunnett's multiple comparisons test (a, b) or one-way ANOVA with Bonferroni posttest (c). *: p<0.05; **: p<0.01; ***: p<0.001; ns: p>0.05. Scale bars, 10 μm.

FIG. 3 shows that K27MH3.3 and G34R pHGG mutants are de novo deposited at damaged replication forks. (a), Graphical representation of the assay to monitor de novo deposition of wildtype H3.3-SNAP at the LacR-occupied LacO array fork barrier in U2OS LacO cells stably expressing SNAP-tagged H3.3 and transfected with mCherry-Lac repressor (LacR). (b), New H3.3-SNAP accumulation at LacR-occupied LacO array analyzed in EdU⁺ and EdU⁻ cells. Data is from five independent experiments, with n>20 for each experiment. (c), Western blot analysis of input and capture samples from iPOND experiments performed in U2OS cells stably expressing wildtype H3.3-SNAP, asynchronous or synchronized in S phase, untreated or treated with camptothecin (3 h, 1 μM). Click-, negative control (no biotin). (d), Schematic representation of the SNAP-PLA assay to visualize the colocalization of YH2A.X with newly synthesized SNAP-tagged H3.3 (labeled with biotin) at RFs damaged with camptothecin (3 h, 0.1 μM). Quantification of SNAP-PLA colocalization foci between new H3.3 and yH2A.X in EdU+U2OS cells stably expressing wildtype (WT_1) or mutant H3.3-SNAP, or SNAP tag only as a control. Mean+s. e. m from up to seven independent experiments, with n>130 for each experiment. Statistical significance is calculated by unpaired t-test with Welch correction (b), one-way ANOVA with Bonferroni posttest (d) *: p<0.05; **: p<0.01; ***: p<0.001; ns: p>0.05. Scale bars, 10 μm.

FIG. 4 shows that abnormal PNKP function in K27MH3.3 and G34R mutant cells mediates aberrant NHEJ and represents a therapeutic target in pediatric pHGG. (a), Identification of proteins associated with wildtype (WT) and mutant H3.3 (K27M, G34R) by proximity-dependent biotinylation (BioID) in HEK293 cells expressing BirA *-tagged H3.3 proteins. Volcano plots show interactors enriched or depleted in K27M (left) or G34R (right) H3.3 samples compared to wildtype H3.3, with each dot representing an interactor. Significant interactors whose log 2 fold change is >1 and whose p-value is <0.05 are visible. Common interactors between K27M and G34R are shown in dark grey (both enriched or depleted ones). Positive controls (EZH2, SUZ12 and NSD1) are indicated. (b), Western blot analysis of input and capture samples from iPOND experiments performed in U2OS cells expressing wildtype (WT_1) or mutant H3.3-SNAP, synchronized in S phase and damaged with camptothecin (1 h, 1 μM). Click-, negative control (no biotin). (c), Analysis of NHEJ activity by Random Plasmid Integration Assay (RPIA) in U2OS cells stably expressing wildtype (WT_1) or mutant H3.3 and transfected with siRNAs against Luciferase (siLUC, control) or PNKP (siPNKP). Western blot analysis shows siRNA efficiency (Tubulin, loading control). Vector-, negative untransfected control. (d), Proliferation curves of S. pombe strains expressing mutant H3, depleted for the core NHEJ factor xrc4 (Axrc4) and for pnk1 (Apnk1) and grown in the presence of camptothecin (5 μM). (e), Proliferation assays in patient-derived pHGG cell lines harboring wildtype or mutant H3.3 and transfected with siRNAs against Luciferase (siLUC, control) or PNKP (siPNKP_1 and siPNKP_2). The representative western blots show siRNA efficiencies (Tubulin, loading control). Statistical significance is calculated by one-way ANOVA with Tukey's posttest (c) or by comparing proliferation curves based on non-linear regression with a polynomial quadratic model (e), *: p<0.05; **: p<0.01; ***: p<0.001; ns: p>0.05.

FIG. 5 shows the effect of PNKP knockdown on cell proliferation in (a) non-cancerous astrocytes, (b) patient-derived pHGG cell line harboring H3.3 K27M and mutations in ATRX and TP53 genes, (c) U2OS cells stably expressing H3.3 G34R treated with the indicated siRNAs (siLUC: control siRNA against luciferase) and co-transfected with the indicated plasmids. Western blots show the efficiency of PNKP knockdown and the expression of exogenous GFP-tagged proteins (Tubulin, loading control). The graphs show mean values+/−s. e. m. from four independent experiments (two for NEM 375). *: p<0.05; **: p<0.01; ns, non-significant.

### EXAMPLES

### Example 1

### 1. Material and Methods

### 1.1. Human Cell Lines

U2OS (human osteosarcoma, female, American Type Culture Collection ATCC HTB-96) and Hela cells (human cervical carcinoma, female, ATCC CCL-2) were cultured in Dulbecco's modified Eagle's medium DMEM Gluta-Max (Life Technologies) supplemented with 10% fetal bovine serum (Eurobio) and antibiotics (100 U/ml penicillin, 100 μg/ml streptomycin, Life Technologies) and maintained at 37° C. under 5% CO2 in a humified incubator. U2OS cells stably expressing SNAP-tagged wild-type or mutant H3.3, and U2OS LacO H3.3-SNAP cells with integrated 256 tandem LacO repeats and stably expressing SNAP-tagged wild-type H3.3 (Adam S. et al, 2016) were cultured in the same medium supplemented with 100 μg/ml G418 (Life Technologies).

### 1.2. Generation of U2OS Stable Cell Lines

U2OS cells stably expressing C-terminal, SNAP-tagged H3.3, either wild-type, K27M, G34R, G34V, G34W or K36M were generated by transfection of plasmid encoding wild-type or mutated H3.3 and selection of clones in limiting dilution in medium supplemented with G418 (Life Technologies) starting 48 hours after transfection. To verify the presence of mutations in the clones, genomic DNA was extracted and subjected to PCR amplification with the following primers: 5′-TGGCAGTACATCTACGTATTAGTCA-3′ (SEQ ID NO:5, upstream of the CMV promoter) and 5′-GCTGGTGAAAGTAGGCGTTG-3′ (SEQ ID NO:6, N-terminal to SNAP). The amplification product was verified by Sanger sequencing (GATC Biotech). Single clones harboring each H3.3 mutation were expanded and evaluated for levels of expression of the exogenous H3.3 proteins and for the presence of histone PTM alterations described in tumor samples. The mutant to wild-type H3.3 ratio was evaluated by western blot.

### 1.3. Primary Pediatric Human Glioma Cell Lines

SF9402 and SF9427 (wild-type H3.3) cell lines were cultured as previously reported (Haschizume R et al, 2014). SU-DIPG-XVII and HSJD-002-GBM were cultured in Tumor Stem Medium (Nagaraja S. et al, 2017), which contains DMEM/F12 1:1 (Invitrogen), Neurobasal-A (Invitrogen), 10 mM HEPES (Invitrogen), 1×MEM sodium pyruvate (Invitrogen), 1×MEM non-essential amino acids (Invitrogen), 1% GlutaMax (Invitrogen), 20 ng/ml human basic fibroblast growth factor (CliniSciences), 20 ng/ml human epidermal growth factor (CliniSciences), 20 ng/ml human platelet-derived growth factor (PDGF)-A and PDGF-B (CliniSciences), 10 ng/ml heparin (StemCell Technologies), and 1×B27 without Vitamin A (Invitrogen). DIPG lines were generally grown in suspension flasks as tumorspheres, except when they underwent transfection and proliferation assay, for which they were dissociated and plated on plates coated with laminin (10 μg/mL, Sigma-Aldrich). MGBM1 cells (H3.3G34R) were cultured in DMEM Gluta-Max supplemented with 10% FBS and antibiotics (100 U/ml penicillin, 100 μg/ml streptomycin). All glioma cells were maintained at 37° C. under 5% CO2 in a humified incubator and verified for expression of the expected H3.3 proteins by Western blot analysis with antibodies raised against H3.3K27M or G34R (see Antibody list for details).

### 1.4. Drug Treatments and Inhibitors

Camptothecin (CPT, Sigma-Aldrich) was used at 0.1 μM for 3 h, or at 1 μM for 1 or 3 h for iPOND in human cells, and at 5 or 10 μM in yeast cells; hydroxyurea (HU, Sigma-Aldrich) at 2 mM for 3 h; mitomycin C (MMC, Sigma-Aldrich) at 200 ng/ml and 25 ng/ml for 24 h for repair foci analyses and metaphase spreads, respectively; bleomycin (Bleo, Sigma-Aldrich) at 20 g/mL for 3 h. An overnight treatment with 2 mM Thymidine followed by 3 h release in fresh medium was used to enrich cells in S phase for iPOND experiments (70-75% of cells were in S phase as evaluated by FACS). The EZH2 inhibitor GSK126 (EZH2i, Selleckchem) was used at 1 μM for 72 h.

1.5. Plasmids Used in this Study

The H3F3A and H3F3B human cDNA sequences (GenScript) were cloned by using Clal and EcoRI restriction enzymes into the pSNAPm plasmid (New England Biolabs), with the SNAP tag in the C-terminus of the insert (Table 1). These plasmids were subjected to directed mutagenesis to introduce the cancer-associated mutations (see Table 2 for details of the mutations, primers used and genes involved). Generation of the mutated plasmids was verified by Sanger sequencing (GATC Biotech).

TABLE 1
Plasmids used in this study

| Plasmid | Construct details |
|---|---|
| H3.3-SNAP | Human H3F3A coding sequence cloned |
| | into pSNAPm (NEB) |
| H3.3-SNAP | Human H3F3B coding sequence cloned |
| | into pSNAPm (NEB) |
| pEGFP-C1-IRES-puro | Bicistronic vector coupling EGFP and |
| | puromycin expression |
| mCherry-lacR | mCherry-lacR-NLS (Soutoglou et al, |
| | 2008) |

TABLE 2
Point mutations in H3.3 coding genes: primers used and associated cancers
(primer sequences listed as SEQ ID NO: 7-16)

| Amino- | | | | | |
|---|---|---|---|---|---|
| acid | Mutated | Affected | | Position | |
| mutation | codon | gene | Cancer type | in CDS | Mutagenesis primer sequences |
| K27M | AAG→ATG | H3F3A | Pediatric High | 83 | F: |
| | | | Grade Glioma | | 5′-caaaagccgctcgcatgagtgcgccctctactg-3′ |
| | | | (pHGG) | | R: |
| | | | (Weinberg, D. | | 5′cagtagagggcgcactcatgcgagcggcttttg-3′ |
| G34R | GGG→AGG | H3F3A | N. et al 2017) | 103 | F: |
| | | | | | 5′-gcgccctctactggaagggtgaagaaacctcatc-3′ |
| | | | | | R: |
| | | | | | 5′-gatgaggtttcttcacccttccagtagagggcgc-3′ |
| G34V | GGG→GTG | H3F3A | | 104 | F: |
| | | | | | 5′-gcgccctctactggagtggtgaagaaacctcatc-3′ |
| | | | | | R: |
| | | | | | 5′-gatgaggtttcttcaccactccagtagagggcgc-3′ |
| G34W | GGG→TGG | H3F3A | Giant Cell | 103 | F: |
| | | | Tumor of Bone | | 5′-gcgccctctactggatgggtgaagaaacctcatc-3′ |
| | | | (Weinberg, D. | | R: |
| | | | N. et al 2017) | | 5′-gatgaggtttcttcacccatccagtagagggcgc-3′ |
| K36M | AAG→ATG | H3F3B | Chondroblastoma | 110 | F: |
| | | | (Weinberg, | | 5′-ctaccggcggggtgatgaagcctcatcgctac-3′ |
| | | | D. N. et al | | R: |
| | | | 2017) | | 5′-gtagcgatgaggcttcatcaccccgccggtag-3′ |

F = Forward, R = Reverse, CDS = Coding DNA Sequence.

### 1.6. Immunofluorescence, Image Acquisition and Analysis

Cells grown on glass coverslips (VWR) were either fixed directly with 2% paraformaldehyde (PFA) and permeabilized with 0.2% Triton X-100 in PBS or pre-extracted before fixation with 0.5% Triton X-100 in CSK buffer (Cytoskeletal buffer: 10 mM PIPES pH 7.0, 100 mM NaCl, 300 mM sucrose, 3 mM MgCl₂) to remove soluble proteins (not bound to chromatin) and then fixed with 2% PFA. Samples were blocked in 5% Bovine Serum Albumin (BSA, Sigma-Aldrich) in PBS supplemented with 0.1% Tween 20 (Euromedex) before incubation with primary antibodies and secondary antibodies conjugated to Alexa Fluor 488 or 568 (Invitrogen). Coverslips were mounted in Vectashield medium with DAPI (Vector Laboratories) and observed with a Leica DMI6000 epifluorescence microscope using a Plan-Apochromat 40×/1.3 or 63×/1.4 oil objective. Images were captured using a CCD camera (Photometrics) and Metamorph software. Images were mounted with Adobe Photoshop applying the same treatment of fluorescence levels to all images from the same experiment. Fiji software was used for image analyses. Nuclei were delineated based on DAPI staining. S phase, replicating cells were discriminated based on EdU staining. The position of the LacO array was determined based on mCherry-LacR signal. DNA repair and PLA foci were identified and counted by using the find maxima function (Fiji software), on maximum intensity z-projections in the case of PLA foci. At least 70 cells/sample were scored in each experiment. Results of automatic foci counting were graphed as number of foci per cell or as number of cells with more than 5 or 10 DNA repair foci that was set as a threshold.

### 1.7. Ethynyl-deoxyUridine (EdU) Labeling of S Phase Cells

For discrimination of S phase cells, 10 μM 5-Ethynyl-2′-deoxyUridine (EdU, Sigma-Aldrich) was incorporated into cells for 15 minutes prior to DNA damage treatment and fixation. EdU was revealed using Click-It EdU Imaging kit (Invitrogen) according to manufacturer's instructions.

### 1.8. Random Plasmid Integration Assay (RPIA)

Cells grown in 6-well plates were transfected with siRNAs and, later the same day, the cells were transfected with 2 μg/well gel-purified Fspl-BspDI-linearized pEGFP-C1-IRES-puro plasmid (bicistronic vector coupling EGFP and puromycin expression). The cells were transfected once more with siRNAs the following day. Cells were collected 48 h later, counted and seeded in 10 cm diameter dishes either lacking or containing 0.375 μg/mL puromycin. The transfection efficiency was determined on the same day by FACS analysis of EGFP-positive cells. The cell dishes were incubated at 37° C. to allow colony formation and medium was refreshed on day 4 and 8. On day 10-12, the cells were stained with 0.5% Crystal Violet (Sigma-Aldrich)/20% ethanol solution to score colonies with more than 50 cells. Random plasmid integration events on the puromycin-containing plates were normalized to the plating efficiency (plate without puromycin) and to the transfection efficiency.

### 1.9. Fission Yeast Strains and Genetic Analyses

Schizosaccharomyces pombe strains containing point mutations in histone H3, K27M in hht2+, G34R and G34V in hht3+, were generated by a PCR-based module method. pnk1Δ and xrc4Δ strains were derived from the fission yeast deletion library and the gene deletions were verified by PCR. All other strains were constructed through genetic crosses. For serial dilution plating assays (spot assays), ten-fold dilutions of a mid-log phase culture were plated on the indicated medium and grown for 3 days at 30° C. Overnight liquid S. pombe cultures were grown to saturation in YES media. Saturated cultures were equilibrated to an OD600 of 1.0, arrayed in a 96 well microtiter plate, and pinned in quadruplicate to achieve a 384 colony density (i.e. 4 technical replicates for each position of the microtiter plate) using a Singer RoToR robot (Singer Instruments, Inc. Somerset UK). Strains were grown on YES solid agar media with camptothecin concentrations indicated in the figure legends. Plates with pinned colonies were incubated at 30° C. and scanned every 96 minutes for growth curves by measuring colony density. Expected fitness of a double mutant, ab(E^{ab}) was calculated as the multiplicative fitness contributions of each single mutant (F^{a}, and F^{b}) scaled to fitness of wildtype (F^{wt}=1).

$${E}^{ab} = {F}^{a} \cdot {F}^{b} \pm {ε}^{ab}$$

Error in expected fitness (ε^{ab}) was computed by propagating error from estimates of F^{a}, and F^{b} using the equation below. Care was taken to minimize systematic bias in experiments (e.g. by distributing strains evenly throughout the 96 well plate to minimize position and neighboring strain effects).

$${ε}^{ab} = {E}^{ab} \cdot \sqrt{{( \frac{{ε}^{a}}{{F}^{a}} )}^{2} + {( \frac{{ε}^{b}}{{F}^{b}} )}^{2}}$$

### 1.10. Metaphase Spreads

To prepare metaphase spreads, Colcemid (Gibco) was added to the culture medium at 0.1 μg/ml for 3 h before collecting the cells. Cells were washed in PBS and resuspended in 75 mM KCl for 15 min at 37° C. Cells were then fixed with fresh methanol/acetic acid (v/v=3:1) at −20° C. for at least 16 hours. Cell pellets were further washed with fresh fixative before dropping onto slides. Chromosomes were stained with 5% Giemsa (Gibco) before mounting. Mytomycin (MMC) is added for 24 hours before fixing and harvesting at a concentration of 25 ng/ml. At least 30 metaphase spreads were scored per sample in each experiment for the presence of radial chromosomes.

1.11. Mutational Signature Analysis on Primary pHGG Samples

pHGG samples for single nucleotide variant (SNV) mutational signature analysis were acquired from previously published data available under EGAS00001000575, EGAS00001001139, EGAS00001000572 and EGAS00001000192. Novel data was generated from samples obtained from the DIPG-BATs clinical trial (NCT01182350), the Dana-Farber Tissue Bank or collaborating institutions, under protocols approved by the institutional review board of the Dana-Farber/Harvard Cancer Center with informed consent (DFCI protocols 10417, 10201 and DFCI 19293). DNA was extracted from single Diffuse Midline Glioma cores, pHGG biopsies and autopsy samples using Qiagen AllPrep DNA/RNA extraction kits. For whole-genome sequencing, genomic DNA was fragmented and prepared for sequencing to 60× depth on an Illumina HiSeq 2000 instrument. Reads from both novel and published data were aligned to the reference genome hg19/GRCh37 with BWA83, duplicate-marked, and indexed using SAMtools and Picard. Base quality score was bias adjusted for flowcell, lane, dinucleotide context, and machine cycle and recalibrated, and local realignment around insertions or deletions (indels) was achieved using the Genome Analysis Toolkit. SNV signature analysis was performed using Palimpsest on a VCF containing somatic mutations identified by Mutect2.

### 1.12. Extraction of Cellular Proteins and Western Blot Analysis

Total extracts were obtained by scraping cells in Laemmli buffer (50 mM Tris HCl pH 6.8, 1.6% Sodium Dodecyl Sulfate (SDS), 8% glycerol, 4% β-mercaptoethanol, 0.0025% bromophenol blue) followed by 5-10 min denaturation at 95° C.

For western blot analysis, extracts along with molecular weight markers (Precision plus protein Kaleidoscope standards, Bio-Rad) were run on 4%-20% Mini-PROTEAN TGX gels (Bio-Rad) in running buffer (200 mM glycine, 25 mM Tris, 0.1% SDS) and transferred onto nitrocellulose membranes (Protran) with a Trans-Blot SD semi-dry or wet (Bio-Rad) transfer cell. Proteins of interest were probed using the appropriate primary and Horse Radish Peroxidase (HRP)-conjugated secondary antibodies (Jackson Immunoresearch), detected using SuperSignal West Pico or Femto chemiluminescence substrates (Pierce). The resulting signal was visualized on hyperfilms MP (Amersham) with a film processor (SRX105, Konica).

Primary antibodies were:

| Antibody target | Species | Supplier (Reference) | Dilution | Application |
|---|---|---|---|---|
| RAD51 | Rabbit | Santa Cruz Biotechnology | 1:500 | WB |
| | | sc-8349 (Clone H-92) | 1:250 | IF |
| 53BP1 | Rabbit | Novus Biologicals (NB100-304) | 1:1000 | WB |
| | | | | IF |
| Biotin | Rabbit | Bethyl Laboratories (BETA150- | 1:500 | PLA |
| | | 109A) | | |
| FANCD2 | Rabbit | Novus Biologicals (NB100-182) | 1:500 | WB |
| | | | | IF |
| YH2A.X | Rabbit | Cell Signaling Technology (2577) | 1:1000 | WB |
| YH2A.X | Mouse | EMD Millipore (05-636) | 1:1000 | IF |
| | | | | PLA |
| H3.3 | Rabbit | EMD Millipore (09-838) | 1:1000 | WB |
| H3.3 K27M | Rabbit | EMD Millipore (ABE419) | 1:1000 | WB |
| H3.3 G34R | Rabbit | Cambridge Research Biochemicals | 1:250 | WB |
| | | (crb2005185f) | | |
| H3.3 G34V | Rabbit | Cambridge Research Biochemicals | 1:1000 | WB |
| | | (crb2005186f) | | |
| H3.3K36M | Rabbit | RevMab (31-1085-00) | 1:1000 | WB |
| PCNA | Mouse | Dako (M0879) | 1:000 | WB |
| PNKP | Rabbit | Sigma-Aldrich (HPA006782) | 1:500 | WB |
| SNAP | Rabbit | Pierce Antibodies | 1:500 | WB |
| | | (CAB4255) | 1:1000 | IF |
| CAF-1 p60 | Rabbit | Abcam (ab109442) | 1:1000 | WB |
| XRCC4 | Rabbit | Sigma-Aldrich (HPA006801) | 1:500 | WB |
| H3K27me3 | Rabbit | Cell Signalling (9733) | 1 :250 | WB |
| H3K36me3 | Rabbit | Abcam (ab9050) | 1:2000 | WB |
| H3 | Rabbit | Abcam (ab1791) | 1:1000 | WB |
| RNF168 | Rabbit | EMD Millipore (ABE367) | 1:1000 | WB |
| SETD2 | Rabbit | Abcam (ab31358) | 1:250 | WB |
| Tubulin | Mouse | Sigma-Aldrich (T9026) | 1:10000 | WB |
| RAD51 | Rabbit | Santa Cruz Biotechnology | 1:500 | WB |
| | | sc-8349 (Clone H-92) | 1:250 | IF |
| 53BP1 | Rabbit | Novus Biologicals (NB100-304) | 1:1000 | WB |
| | | | | IF |
| Biotin | Rabbit | Bethyl Laboratories (BETA150-109A) | 1:500 | PLA |
| FANCD2 | Rabbit | Novus Biologicals (NB100-182) | 1:500 | WB |
| | | | | IF |
| YH2A.X | Rabbit | Cell Signaling Technology (2577) | 1:1000 | WB |
| YH2A.X | Mouse | EMD Millipore (05-636) | 1:1000 | IF |
| | | | | PLA |
| H3.3 | Rabbit | EMD Millipore (09-838) | 1:1000 | WB |
| H3.3 K27M | Rabbit | EMD Millipore (ABE419) | 1:1000 | WB |
| H3.3 G34R | Rabbit | Cambridge Research Biochemicals | 1:250 | WB |
| | | (crb2005185f) | | |
| H3.3 G34V | Rabbit | Cambridge Research Biochemicals | 1:1000 | WB |
| | | (crb2005186f) | | |
| H3.3K36M | Rabbit | RevMab (31-1085-00) | 1:1000 | WB |
| PCNA | Mouse | Dako (M0879) | 1:000 | WB |
| PNKP | Rabbit | Sigma-Aldrich (HPA006782) | 1:500 | WB |
| SNAP | Rabbit | Pierce Antibodies | 1:500 | WB |
| | | (CAB4255) | 1:1000 | IF |
| CAF-1 p60 | Rabbit | Abcam (ab109442) | 1:1000 | WB |
| XRCC4 | Rabbit | Sigma-Aldrich (HPA006801) | 1:500 | WB |
| H3K27me3 | Rabbit | Cell Signalling (9733) | 1 :250 | WB |
| H3K36me3 | Rabbit | Abcam (ab9050) | 1:2000 | WB |
| H3 | Rabbit | Abcam (ab1791) | 1:1000 | WB |
| RNF168 | Rabbit | EMD Millipore (ABE367) | 1:1000 | WB |
| SETD2 | Rabbit | Abcam (ab31358) | 1:250 | WB |
| Tubulin | Mouse | Sigma-Aldrich (T9026) | 1:10000 | WB |

Secondary antibodies were:

TABLE 3
Antibodies used

| | | | | |
|---|---|---|---|---|
| Rabbit HRP | Donkey | Jackson | 1:10000 | WB |
| | | Laboratories | | |
| | | 711-035-152 | | |
| Mouse HRP | Goat | Jackson | 1:10000 | WB |
| | | Laboratories | | |
| | | 115-035-068 | | |
| Rabbit Alexa Fluor 488 | Goat | Invitrogen (A11034) | 1:1000 | IF |
| Rabbit Alexa Fluor 568 | Goat | Invitrogen (A11036) | 1:1000 | IF |
| Mouse Alexa Fluor 488 | Goat | Invitrogen (A11029) | 1:1000 | IF |
| Mouse Alexa Fluor 568 | Goat | Invitrogen (A11031) | 1:1000 | IF |

HRP: HorseRadish Peroxidase;
IF: Immunofluorescence;
PLA: Proximity Ligation assay
WB: western blot

1.13.siRNA and plasmid transfections

siRNAs purchased from Eurofins MWG Operon or Sigma-Aldrich (Table 4) were transfected into cells using Lipofectamine RNAiMAX (Invitrogen) following manufacturer's instructions. Cells were analyzed and/or harvested 48 to 72 h post-transfection except for proliferation assays, where cells were analyzed over a 7-day period after transfection.

Cells were transfected with plasmid DNA (see plasmid section) using Lipofectamine 2000 (Invitrogen) according to manufacturer's instructions.

TABLE 4
siRNA sequences (listed as SEQ ID NO: 17-25)

| SIRNA | Target sequence (5′-3′) |
|---|---|
| siFANCD2 | CCATGTCTGCTAAAGAGCGTTCATT |
| siH3.3 | 1:1 combination of CTACAAAAGC |
| | CGCTCGCAA (H3F3A) and |
| | GCTAAGAGAGTCACCATCAT (H3F3B) |
| siLUC | CGTACGCGGAATACTTCGA |
| (Luciferase control) | |
| siPNKP_1 | CCGGATATGTCCACGTGAA |
| SiRNF168 | GGCGAAGAGCGATGGAGGA |
| siSETD2 | GTGAAGGAGTATGCACGAA |
| siPNKP_2 | GGAAACGGGTCGCCATCGA |
| siXRCC4 | ATATGTTGGTGAACTGAGA |

### 1.14. SNAP Labeling of Newly Synthesized Histones.

For labeling newly synthesized SNAP-tagged histones (Bodor et al, 2012), parental histones were quenched with 10 μM SNAP-cell Block (NEB) for 30 minutes in culture medium followed by 30-min wash in fresh medium and a 2-h chase. To mark S phase cells/replication forks, EdU was incorporated for 15 minutes just before the quench step. The new SNAP-tagged histones synthesized during the chase were fluorescently labelled with 4 μM of the green-fluorescent reagent SNAP-cell Oregon green (New England Biolabs) during a 30-min pulse step followed by 30-min wash in fresh medium. Alternatively, when combined with Proximity Ligation Assay (PLA), new SNAP-tagged histones were pulse-labeled for 30 min with 5 nM SNAP-biotin (New England Biolabs) diluted 1:200 in 10% Duolink blocking buffer (Sigma-Aldrich) in PBS. After washings, soluble proteins were removed by permeabilization with 0.5% Triton X-100 in cytoskeleton (CSK) buffer, and cells were fixed and processed for immunostaining or PLA.

### 1.15. Proximity Ligation Assay (PLA)

PLA (Söderberg O. et al, 2006) was performed to detect colocalization foci between newly synthesized H3.3-SNAP and yH2A.X at camptothecin-damaged replication forks. The Duolink® In Situ PLA® detection kit (Sigma) was used following manufacturer's recommendations. Briefly, cells on glass coverslips (VWR) were incubated 1 h at 37° C. in Duolink blocking buffer (Sigma-Aldrich) and then for 1 h at room temperature with a mix of the two primary antibodies directed against the target proteins (anti-biotin to detect new H3.3-SNAP-biotin and anti-yH2A.X to detect sites of DNA damage) diluted in antibody dilution reagent (Sigma-Aldrich). Coverslips were then incubated for 1 h at 37° C. with secondary antibodies each harboring a PLA probe (Duolink In Situ PLA MINUS/PLUS probes, Sigma-Aldrich). The PLA probes that bind to the constant regions of the primary antibodies contain a unique DNA strand. If the proteins of interest interact with each other, the DNA probes hybridize to make circular DNA during the 30 min ligation step at 37° C. The resulting circle DNA can be amplified (1 h 40 min amplification at 37° C., Duolink In Situ Detection Reagents Green, Sigma-Aldrich) and visualized by fluorescently labeled complementary oligonucleotide probes incorporation. Coverslips were mounted in Duolink In Situ Mounting Medium with DAPI (Sigma-Aldrich). To study PLA foci in S phase cells, EdU labeling by click chemistry was performed before the blocking step.

1.16. Isolation of Proteins on Nascent DNA (iPOND)

iPOND was performed largely as described previously (Sirbu B. M. et al, 2012), with the following modifications. A total of 3×10⁷ logarithmically growing cells per sample were labeled with 10 μM EdU for 15 min. Following EdU incorporation, cells were fixed with 1% formaldehyde for 15 min at room temperature, followed by 5-min incubation with 0.125 M glycine to quench the formaldehyde. Cells were harvested by scraping, washed three times with PBS, flash frozen in liquid nitrogen and kept at −80° C. Within two weeks, samples were processed for EdU-based pulldown and purification of replication fork-associated proteins. Briefly, click chemistry reactions were performed on pre-permeabilized samples to conjugate biotin to the EdU-labeled DNA by using Biotin Picolyl azide (Sigma Aldrich). Sonication was performed with a Bioruptor Pico sonicator (Diagenode) and DNA shearing was evaluated on an agarose gel. Shearing for optimal detection of the proteins of interest was set to an average DNA fragment size of 800 bp. Total input samples were taken after sonication and clearing of samples and kept at −20° C. until loading on SDS-PAGE gels. Streptavidin beads (Dynabeads MyOne Streptavidin-C1, Life technologies) were used to capture the biotin-conjugated DNA-protein complexes. Captured complexes were washed extensively using SDS and high-salt wash buffers. Purified replication fork proteins were eluted under reducing conditions by boiling in Laemmli sample buffer for 5 min. Total input and capture samples corresponding to equal amounts of cells were resolved on SDS-PAGE gels and analyzed by western blot.

### 1.17. Flow Cytometry and Cell Cycle Analysis

Cells were fixed in ice-cold 70% ethanol before DNA staining with 50 μg/mL propidium iodide (Sigma-Aldrich) in PBS containing 0.05% Tween 20 and 0.5 mg/mL RNase A (USB/Affymetrix). DNA content was analyzed by flow cytometry using a FACSCalibur Flow Cytometer (BD Biosciences) and FlowJo Software (TreeStar).

### 1.18. Human Cell Proliferation Assays

The effect of PNKP knockdown on cell proliferation in human cells was measured as follows: 24 h after siRNA transfection, cells were seeded in 60-mm diameter tissue culture plates (20 000 cells/plate for U2OS, 40 000 to 80 000 for pHGG cells). Cell viability was assessed after 3, 5 and 7 days in culture by staining with trypan blue (Invitrogen) and counting with an automated cell counter (Countess, Invitrogen).

### 1.19. Statistical Analysis

Statistical analyses were carried out using Graphpad Prism software. P values for mean comparisons between two groups were calculated with a Student's t test with Welch's correction when necessary. Multiple comparisons were performed by one- or two-way ANOVA with Bonferroni, Tukey's or Dunnett's post-tests or using the non-parametric Kruskal-Wallis test in case of non-gaussian distributions. Comparisons of proliferation curves were based on non-linear regression with a polynomial quadratic model. ns: non-significant, *p<0.05, ** p<0.01, *** p<0.001, ****: p<0.0001. Statistical parameters including sample size (n) and dispersion of the data (SD or SEM) are indicated in the figure legends.

### 2. Results

### 2.1. H3.3 Mutants Drive Misrepair in S Phase

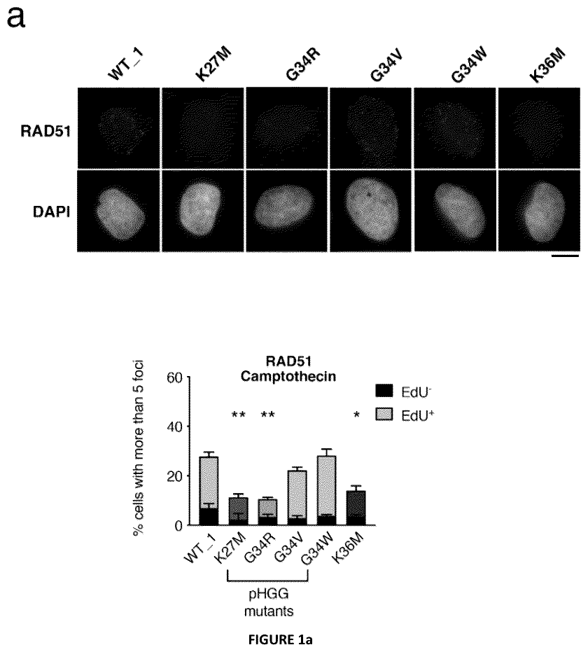

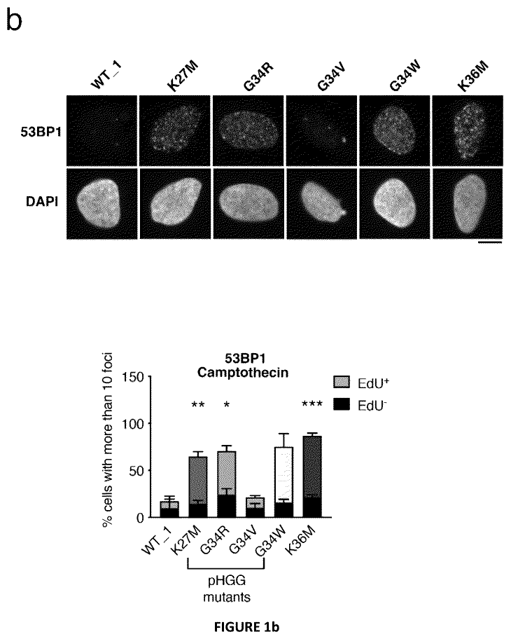

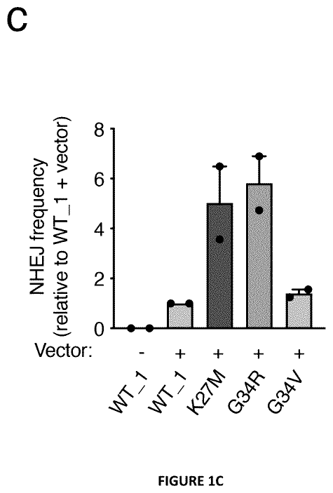

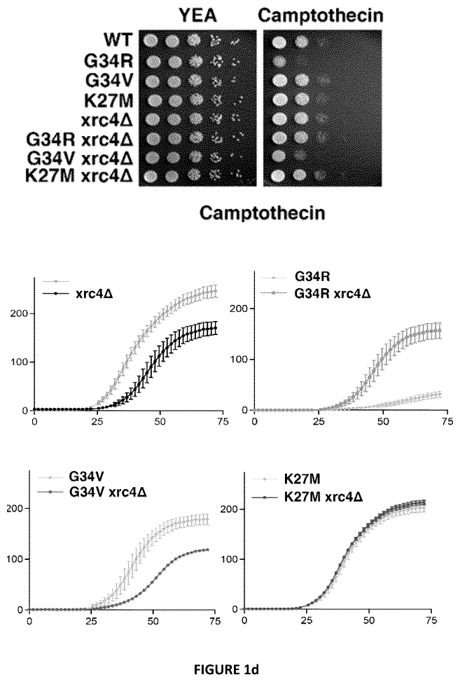

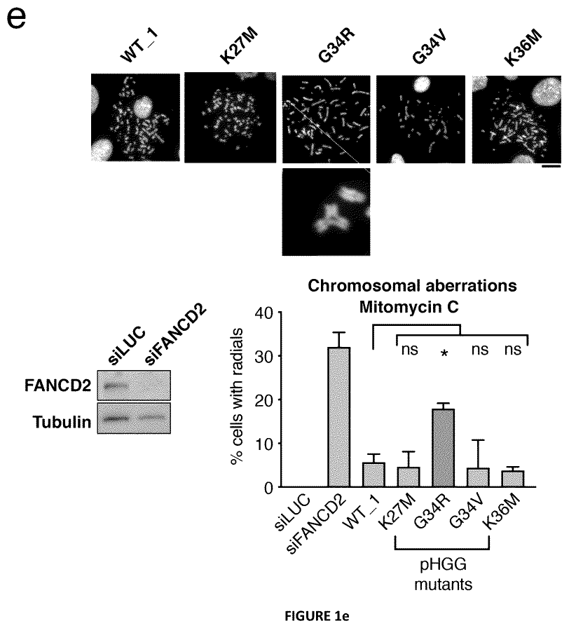

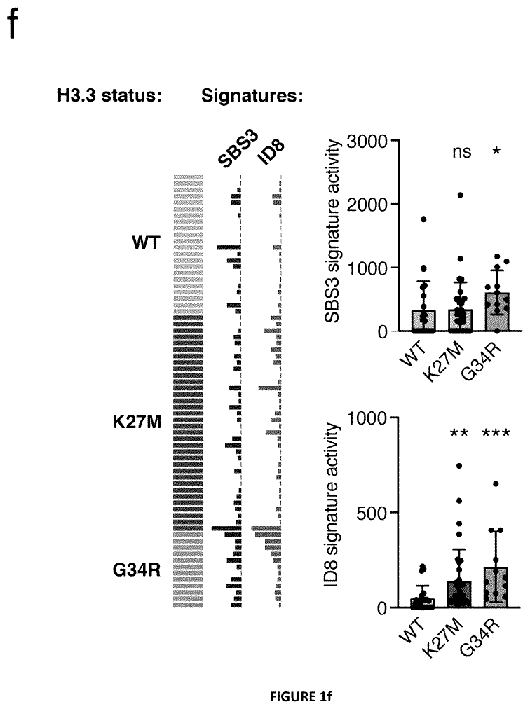

To study the impact of various H3.3 mutations on DNA repair in human cells in an isogenic context, U2OS cell lines stably expressing SNAP-tagged wild-type or individual mutant H3.3 proteins (bearing K27M, G34R/V pHGG mutations, and G34W, K36M non-pHGG mutations) were generated in a wild-type background. The cell lines have comparable expression of the different H3.3-SNAP proteins. They also recapitulate the main histone PTM changes (H3K27me3 and H3K36me39) and the mutant to wild-type H3.3 ratio that characterize H3.3 mutant pHGGs (Weinberg, D. N., 2017). In this system, the focal accumulation of DNA damage response factors upon treatment was analyzed with different genotoxic agents. Similar to the positive control K36M, two pHGG mutants, K27M and G34R, showed impaired foci formation of the recombinase RAD51 Homolog 1 (RAD51), and of Fanconi Anemia Complementation Group D2 (FANCD2) (FIG. 1A), involved in pathways that preferentially repair S phase damage. A possible compensatory activation of the non-homologous end joining (NHEJ) repair pathway was studied in the same cell lines and it was observed increased foci formation of TP53-binding protein 1 (53BP1), a positive regulator of NHEJ. The altered recruitment of repair factors was detectable upon interference with replication fork (RF) progression by camptothecin (CPT), hydroxyurea (HU) or mitomycin C (MMC), but not upon treatment with the radiomimetic agent bleomycin, which triggers DNA damage throughout the cell cycle (FIG. 1A, 1B), indicating that H3.3 K27M and G34R mutants skew the repair of RF-associated DNA lesions. Importantly, the observed defect is not due to a differential cell cycle distribution of cells expressing mutant H3.3 (not shown), to differential induction and signaling of DNA damage, as shown by comparable levels of yH2AX (not shown), nor to differential expression levels of repair proteins (not shown). To functionally analyze NHEJ activity, the Random Plasmid Integration Assay was used, which confirmed increased NHEJ activity in cells expressing the pHGG H3.3 mutants K27M and G34R, but not G34V (FIG. 1C). G34R and G34V histone mutants also play opposing roles in DNA damage repair in fission yeast (Lowe B. R, 2021), prompting to exploit this model system to assess the possible conservation of increased, aberrant NHEJ activity in cells expressing pHGG mutants. Deletion of the core NHEJ factor xrc4 (xrc44) sensitized H3 wildtype and G34V strains to CPT damage, consistent with a protective role of NHEJ in these strains, which was not observed in K27M and G34R strains (FIG. 1D). xrc4 deletion (xrc44) even rescued the CPT sensitivity of G34R yeast cells, indicating that aberrant NHEJ drives the sensitivity of this strain to CPT.

To test whether aberrant DNA repair in H3.3K27M and G34R cells is associated with genome instability in human cells, we examined the occurrence of radials, chromosomal aberrations that derive from misjoining of broken chromatids through aberrant NHEJ. We observed a marked accumulation of radials in H3.3 G34R U2OS cells upon MMC treatment (FIG. 1E). To conclusively link the aberrant DNA repair of H3.3K27M and G34R cells to genome instability onset in a glioma context, whole genome-sequencing data were analyzed from a panel of untreated, p53-mutant primary pHGGs for the presence of mutational signatures. Both H3.3K27M and G34R pHGGs presented higher levels of mutational signatures deriving from aberrant NHEJ (ID8) and defective HR (SBS3) compared to wild-type H3.3 pHGGs (FIG. 1F). Collectively, these data support a model where the pHGG H3.3 mutations K27M and G34R skew the repair of S phase DNA damage towards aberrant NHEJ, thus sustaining a specific pattern of genome instability.

### 2.2. Gain-of-Function DNA Repair Defect

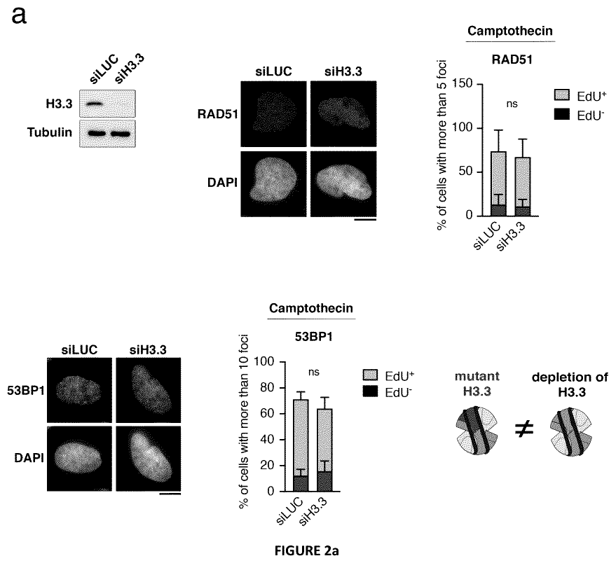

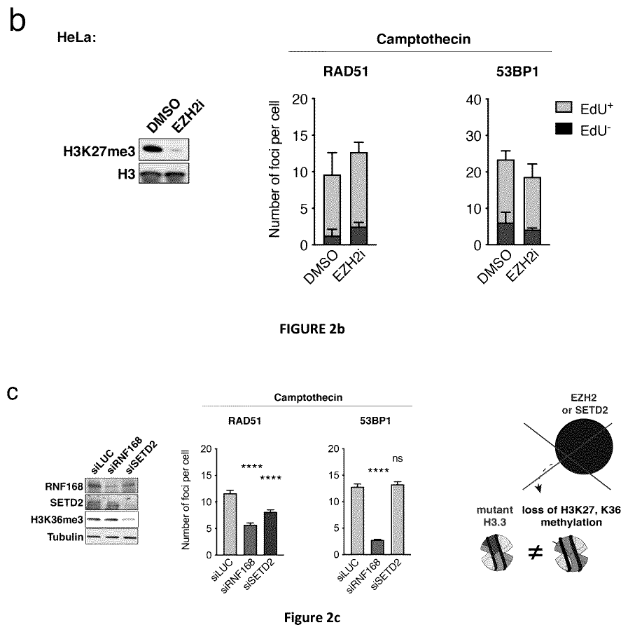

To test whether H3.3 pHGG mutants skew the repair of S phase damage through gain- or loss-of-function mechanisms, the impact of siRNA-mediated depletion of H3.3 was first evaluated on RAD51 and 53BP1 foci formation in CPT-damaged U2OS cells. Depletion of H3.3 did not affect the proportion of cells in S phase (not shown) nor yH2A.X induction in response to CPT (not shown). Contrary to H3.3 mutations, H3.3 loss did not alter RAD51 and 53BP1 foci formation post CPT (FIG. 2A), despite an increase in 53BP1 nuclear levels (not shown). Thus, K27M and G34R mutations on H3.3 do not phenocopy H3.3 loss but rather confer a new function to histone H3.3 upon CPT-induced damage, corroborating the gain-of-function hypothesis.

Dysregulation of gene expression programs by K27M and G34R H3.3 mutations is mediated by reduced trimethylation at lysines 27 and 36 of histone H3 (Weinberg D. N. et al, 2017 & Deshmukh, S. 2021) (H3K27me3 and H3K36me3), respectively. To study whether the observed DNA repair defect is mediated by analogous perturbations of histone PTMs, H3K27 and K36 trimethylation was reduced by inhibiting or depleting the corresponding histone methyltransferases. The H3K27 methyltransferase Enhancer of Zeste 2 (EZH2) is endogenously inhibited in U2OS cells (Ragazzini, R. et al. 2019), thus preventing further reduction of H3K27me3 upon expression of H3.3 K27M (not shown). Yet, aberrant DNA repair was observed in H3.3 K27M U2OS cells (FIG. 1), arguing against a contribution of H3K27me3 reduction to this repair defect. This was confirmed by chemical inhibition of EZH2 (EZH2i) in Hela cells (FIG. 2B), which did not recapitulate the DNA repair defect previously observed upon H3.3 K27M expression. Similarly, reducing H3K36me3 by depleting SET Domain Containing 2 (SETD2) did not result in increased 53BP1 foci formation but solely in the expected reduction of RAD51 foci formation (FIG. 2C). These experiments demonstrate that H3.3 K27M and G34R mutants skew DNA repair in S phase by conferring a gain-of-function to histone H3.3 independent from hypomethylation of H3 K27 and K36.

### 2.3. Mutant H3.3 Deposition at Damaged Forks

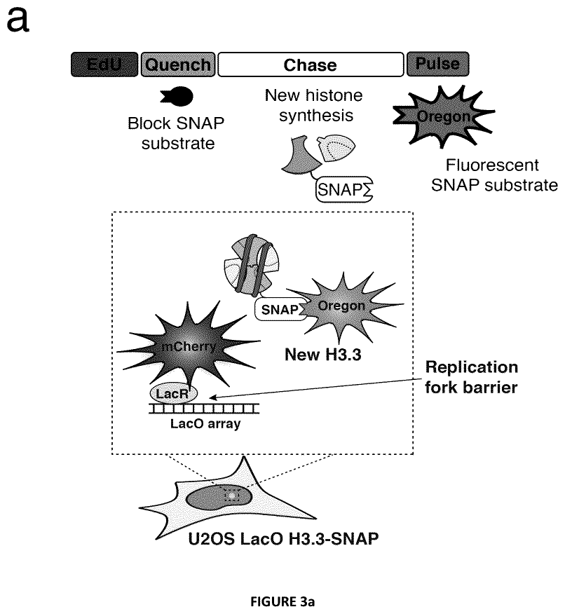

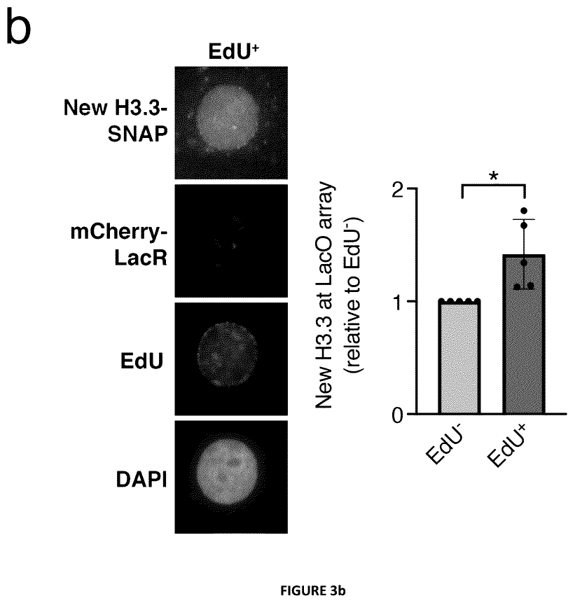

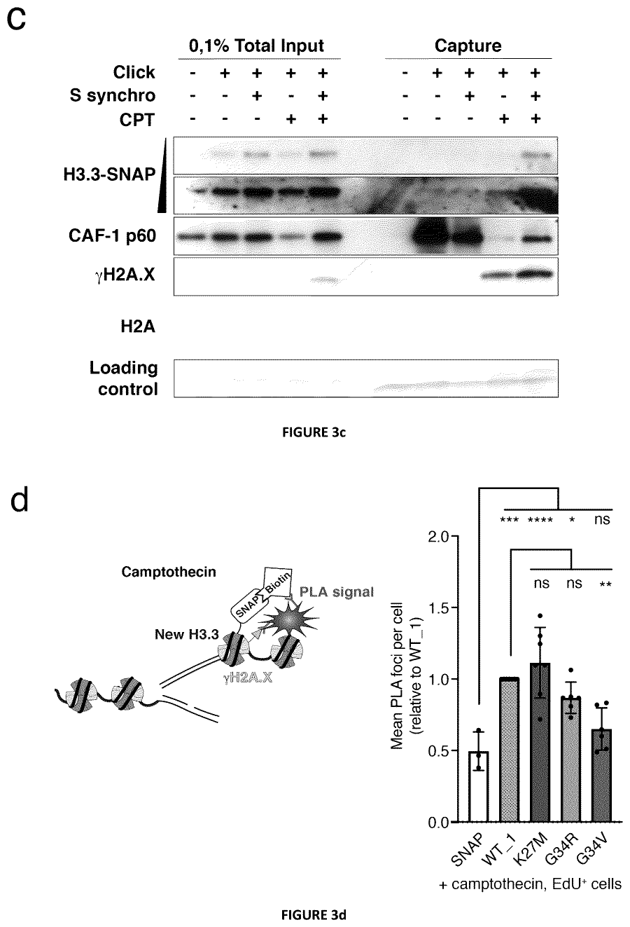

H3.3 de novo deposition at sites of DNA damage (Ferrand J., et al, 2020) prompted us to investigate whether wild-type and pHGG H3.3 mutants were de novo deposited at damaged RFs. First, fluorescent labeling of SNAP-tagged, newly synthesized histone H3.3 was exploited in a model of RF blockage, where stably integrated Lac operon arrays generate an obstacle to DNA polymerase progression when bound by the Lac repressor (LacR). Upon RF blockage, monitored by yH2AX accumulation (FIG. 3A), a local enrichment of fluorescently labeled H3.3 was observed on the array specifically in S phase cells (FIG. 3B), revealing a previously uncharacterized de novo deposition of H3.3 at sites of replication block. These findings were validated through the proteomic-based isolation of proteins at nascent DNA (iPOND45) upon RF damage with CPT in U2OS cells expressing wild-type H3.3-SNAP (FIG. 3C). The enrichment of SNAP-tagged H3.3 at CPT-damaged RFs was enhanced in S phase synchronized cells, supporting an S phase-specific deposition of H3.3 at damaged RFs. The same approach revealed an accumulation of all pHGG H3.3 mutants at CPT-damaged RFs. To further study the deposition of newly synthesized H3.3 mutants, a novel imaging-based method was set-up: SNAP-PLA, that measures by Proximity Ligation Assay (PLA) (Söderberg et al, 2006) the colocalization between biotin-labeled, newly synthesized SNAP-tagged histones and yH2AX at CPT-damaged RFs (FIG. 3D, left). Thus, we could detect de novo deposition of wild-type H3.3 specifically in CPT-damaged, S phase cells (FIG. 3D, right), recapitulating data obtained in the LacO system (FIG. 3B) and validating the SNAP-PLA approach. Moreover, de novo deposition of H3.3K27M and G34R mutants was detected at damaged RFs, which was comparable to that of wild-type H3.3, while the de novo deposition H3.3 G34V was slightly reduced (FIG. 3D, right). These findings put forward a new, local function of H3.3 in RF protection and repair and suggest that pHGG H3.3 mutants may locally affect the chromatin landscape and/or the recruitment of repair factors at damaged RFs, ultimately skewing fork repair.

2.4. PNKP Associates with Mutant H3.3

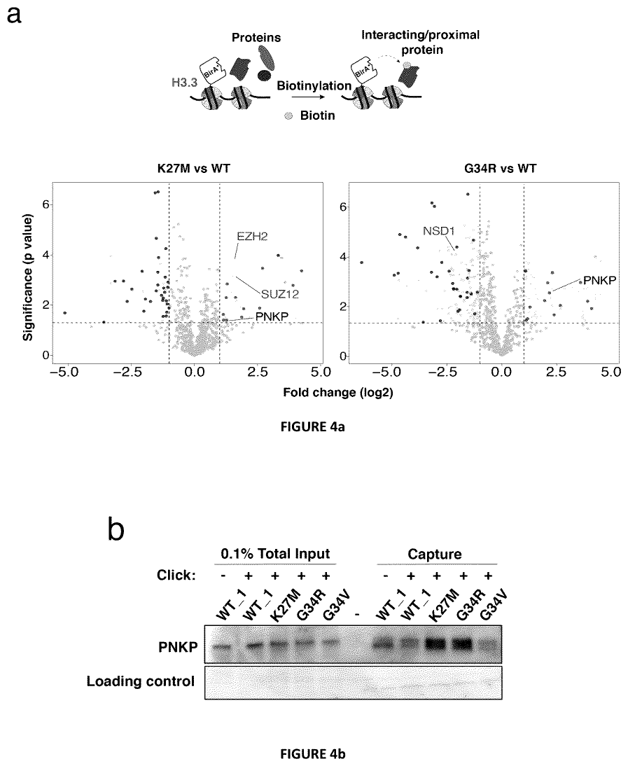

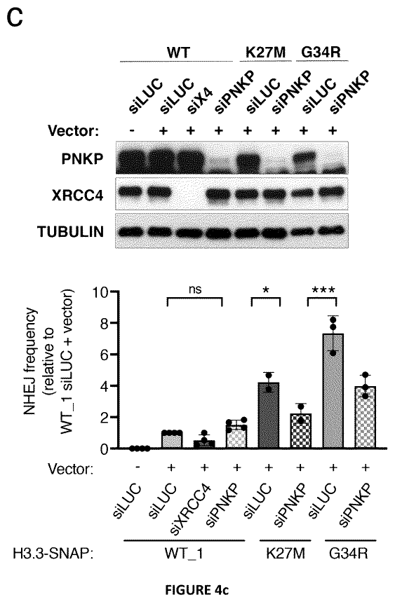

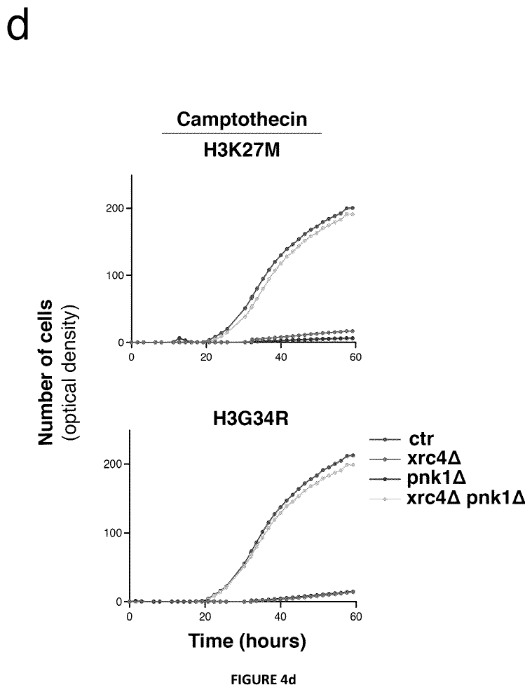

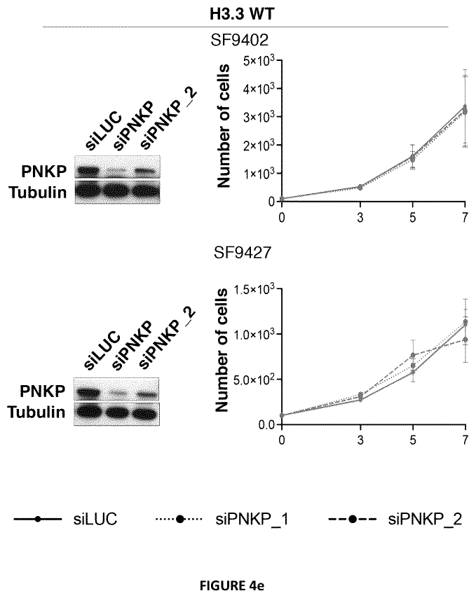

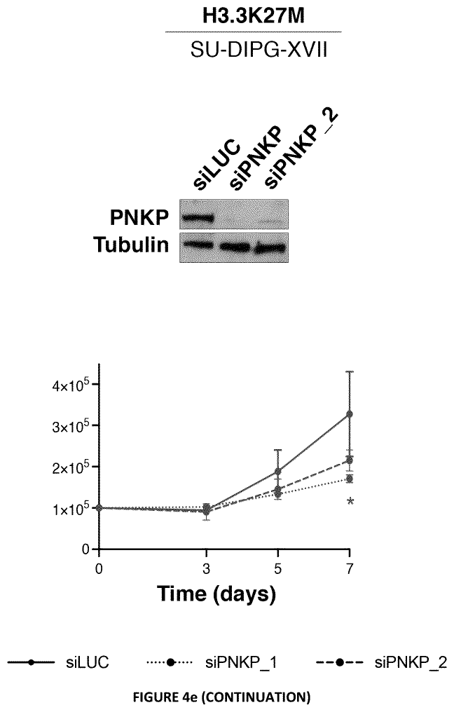

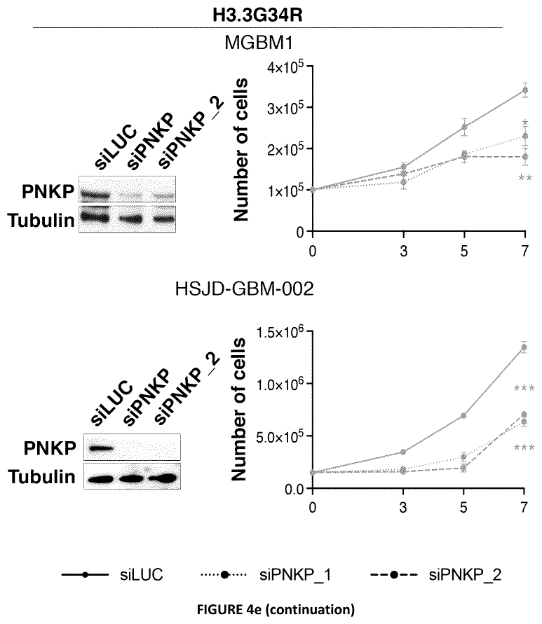

To identify DNA repair factors that preferentially associate with the H3.3K27M and G34R mutants, we employed proximity-dependent biotinylation (BioID) (Scott, W. A. & Campos, E, 2020; Roux et al, 2012) in human cells ectopically expressing wild-type, K27M or G34R H3.3 proteins fused to the mutant BirA* biotin ligase followed by mass spectrometry analysis. Validating this approach, we detected the expected preferential association of EZH2 with the K27M mutant while Nuclear Receptor Binding SET Domain Protein 1 (NSD1), responsible for mono and dimethylation of H3K36, showed reduced association to G34R, in accordance with the reduced methylation of H3K36 by NSD1 in the presence of G34R (FIG. 4A). Among the DNA repair enzymes that preferentially associated with both K27M and G34R compared to wild-type H3.3, we focused our attention on the DNA end processing enzyme Polynucleotide Kinase 3′-Phosphatase (PNKP), which contributes to NHEJ by transferring a phosphate group between broken DNA ends before ligation (Dumitrache, L. C. & Mckinnon, P. J. 2017). Furthermore, PNKP was identified as an H3.3G34R interactor in a previous study (Lim, J. et al. 2018) and plays a central role in neurodevelopment (Dumitrache, L. C. & Mckinnon, P. J. 2017). PNKP total levels were not increased in cells expressing H3.3 K27M or G34R (not shown). However, by iPOND, we observed increased binding of PNKP to CPT-damaged RFs in H3.3K27M and G34R cells (FIG. 4B), further substantiating the preferential association of this DNA repair enzyme with both H3.3 mutants.

### 2.5. PNKP Promotes Misrepair in Mutant Cells

PNKP preferential association with H3.3K27M and G34R may drive the aberrant NHEJ observed in cells expressing these mutants. NHEJ activity was measured by random plasmid integration assay upon knockdown of PNKP in U2OS cells expressing wildtype or mutant H3.3. While PNKP knockdown, differently from XRCC4, does not affect NHEJ activity in wild-type H3.3 cells, it does reduce NHEJ in H3.3K27M and G34R cells, showing that PNKP mediates aberrant NHEJ repair in these cells (FIG. 4C). Similarly, in fission yeast, the rescue of CPT-sensitivity in the G34R strain by xrc4 deletion was not observed upon co-deletion of the PNKP ortholog pnk1, arguing that the aberrant xrc4-mediated NHEJ in a G34R background is dependent on pnk1 (FIG. 4D). Together, these data demonstrate that PNKP drives aberrant NHEJ in H3.3 mutant cells.

2.6. PNKP as a Therapeutic Target in pHGG

The importance of aberrant PNKP function was next assessed in H3.3 mutant cells. PNKP knockdown specifically impaired the growth of H3.3K27M and G34R U2OS cells, but not of wild-type or G34V mutant H3.3 cells (not shown). Similarly, fission yeast strains engineered with K27M and G34R H3 mutations are dependent on pnk1 for proliferation while cells expressing wild-type H3 and the G34V mutant are not (not shown), supporting an evolutionarily conserved functional interaction of K27M and G34R histone mutations with the repair enzyme PNKP. By exploiting a panel of patient-derived glioma cells lines (not shown) and two different siRNAs against PNKP, the specific effect of PNKP knockdown was corroborated on the proliferation of glioma cells harboring endogenous H3.3G34R (MGBM1 and HSJD-002) or H3.3K27M (SU-DIPG-XVII) in contrast to glioma cells with wild-type H3.3 (SF9402 and SF9427), which were unaffected (FIG. 4E). These data expand our findings to state-of-the-art pHGG systems and put forward PNKP as a potential therapeutic target in pHGG cells expressing specific H3.3 mutations.

### DISCUSSION

The present inventor shows that the mutations H3.3K27M and G34R affect RF repair through a mechanism that is distinct from their interference with gene expression programs. The DNA repair defect indeed does not rely on H3K27/K36me3 alterations, but may involve other PTM changes in mutant nucleosomes, possibly through the recruitment of histone modifying enzymes, which may in turn affect the binding of repair factors.

H3.3 G34R and G34V mutants display strikingly opposite DNA repair phenotypes, conserved from yeast to human, the molecular bases of which are still elusive. It is speculated that the bulkier side chain of arginine chain may cause a more drastic disruption of the H3.3 interactome.

The K27M mutation is also found in the H3.1 histone variant in some pHGG (Weinberg D. N. et al, 2017) and shown to inhibit NHEJ in human fibroblasts (Ferrand J., et al, 2020), an opposite phenotype to that of H3.3K27M in U2OS cells. Even if H3.1K27M and H3.3K27M can only be compared if studied in the same cellular background, differences in their DNA repair function can be anticipated since they show distinct distribution patterns in chromatin, present different co-occurring mutations (Ferrand J., et al, 2020) and clinical features in pHGG (Ferrand J., et al, 2020).

### Example 2

### 1. Complementary Material and Methods

### 1.1. Cell Lines

NEM 375 pediatric glioma cell line (H3.3 K27M, p53 and ATRX mutant, GSC12 in (Werbrouck et al., 2019) was grown on laminin in TS medium supplemented with growth factors (NeuroCult NS-A medium with proliferation supplement, Stemcell technologies), heparin (2 μg/mL, Stemcell technologies), human-basic FGF (20 ng/ml, Peprotech), human-EGF (20 ng/ml, Peprotech), PDGF-AA (10 ng/ml, Peprotech), and PDGF-BB (10 ng/ml, Perprotech) (Plessier et al., 2017).

Normal astrocytes CRL-8621 (ATCC) were grown in Eagle's Minimum Essential Medium (EMEM) ATCC® 30-2003 supplemented with 10% fetal bovine serum (Eurobio) and antibiotics (100 U/ml penicillin, 100 μg/ml streptomycin, Life Technologies).

1.2. siRNA

siPNKP 3′UTR:
(SEQ ID NO: 26)
5′-CCACAAUAAACGCUGUUUC-3′.

### 1.3. Plasmids

GFP: pEGFP-C2 (Clontech)

GFP-PNKP: pEGFP-C2-PNKP (Aceytuno et al., 2017)

1.4. siRNA and Plasmid Co-Transfection

For rescue experiments, cells were concomitantly transfected with siRNA (50 nM final) and plasmid DNA (0.5 μg/ml final) using Lipofectamine 2000 (Invitrogen) according to manufacturer's instructions.

### 2. Results

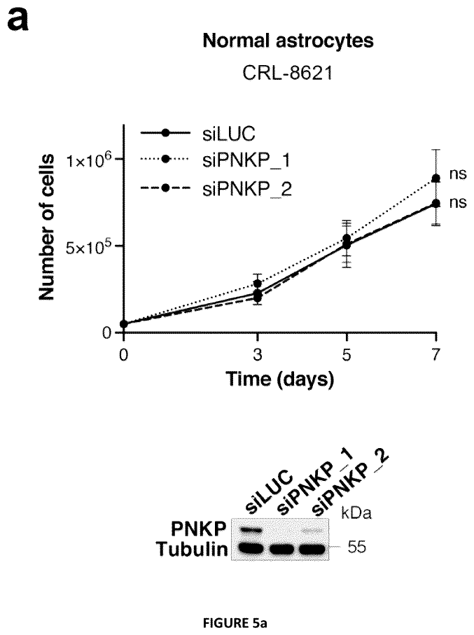

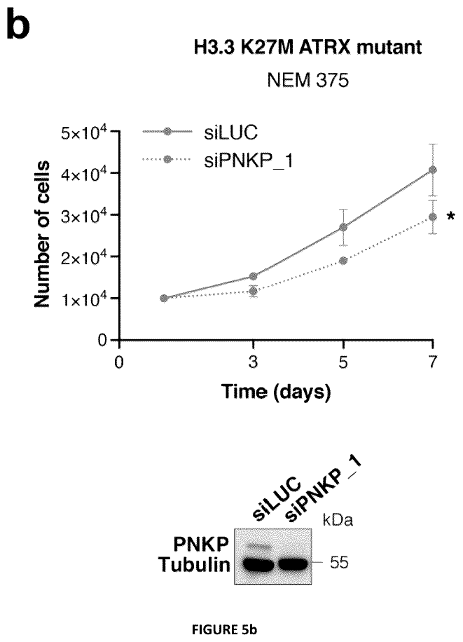

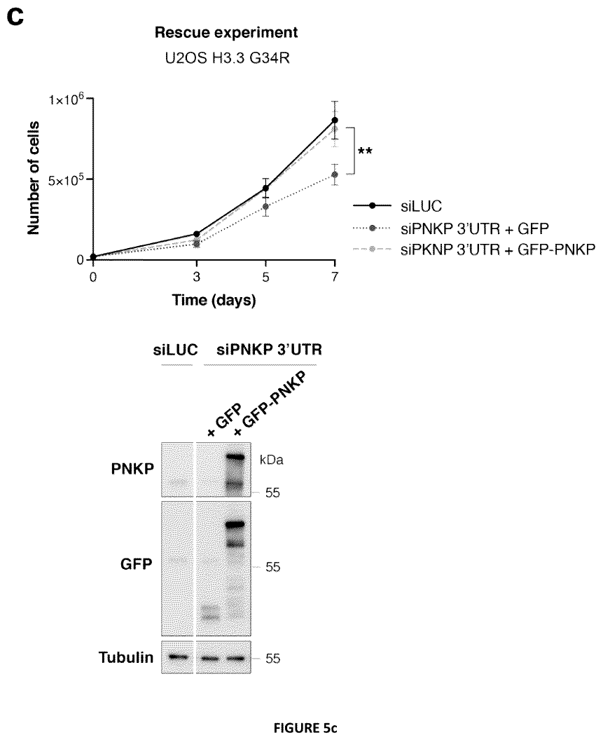

To assess the specificity and potential side effects of PNKP targeting, the inventors evaluated the anti-proliferative effect of PNKP downregulation in normal, non-transformed astrocytes. The inventors observed that PNKP knockdown did not impact cell growth in non-cancerous astrocytes (FIG. 5a). To characterize the genetic determinants that correlate with an optimal anti-proliferative response to PNKP downregulation, it is important to evaluate the effect of PNKP downregulation in pHGG cell lines harboring different mutational status of genes such as ATRX and TP53 that are known to be relevant for pHGG pathogenesis and radiotherapy response (Ferrand et al., 2020; Werbrouck et al., 2019). The inventors thus investigated the impact of PNKP knockdown in an additional H3.3 K27M pHGG cell line, NEM 375 (Werbrouck et al., 2019), which also harbors mutated ATRX and which was sensitive to PNKP knockdown (FIG. 5b), similar to the ATRX wild-type cell line SU-DIPG-XVII. These results indicate that PNKP targeting efficiently impacts pHGG cell growth regardless of their ATRX mutational status.

To conclusively link the anti-proliferative effect to PNKP loss-of-function, the inventors performed rescue experiments and observed that exogenous expression of GFP-tagged PNKP rescued cell growth in PNKP knocked down cells (FIG. 5c).

### BIBLIOGRAPHIC REFERENCES

Aceytuno, R. D., Piett, C. G., Havali-Shahriari, Z., Edwards, R. A., Rey, M., Ye, R., Javed, F., Fang, S., Mani, R., Weinfeld, M., et al. (2017). Structural and functional characterization of the PNKP-XRCC4-LigIV DNA repair complex. Nucleic Acids Res 45, 6238-6251. doi.org/10.1093/nar/gkx275.
Adam, S. et al. Real-Time Tracking of Parental Histones Reveals Their Contribution to Chromatin Integrity Following DNA Damage. Mol. Cell 64, 65-78 (2016).
Bodor, D. L., Rodríguez, M. G., Moreno, N. & Jansen, L. E. T. Analysis of Protein Turnover by Quantitative SNAP-Based Pulse-Chase Imaging. in Current Protocols in Cell Biology vol. Chapter 8 (John Wiley & Sons, Inc., 2012).
Day, C., Grigore, F., Langfald, A., Hinchcliffe, E. & Robinson, J. CBIO-11. HISTONE H3.3 G34R/V MUTATIONS STIMULATE PEDIATRIC HIGH-GRADE GLIOMA FORMATION THROUGH THE INDUCTION OF CHROMOSOMAL INSTABILITY. Neuro-Oncol. 23, vi29 (2021).
Deshmukh, S., Ptack, A., Krug, B. & Jabado, N. Oncohistones: a roadmap to stalled development. FEBS J. (2021) doi: 10.1111/febs.15963.
Dumitrache, L. C. & Mckinnon, P. J. Polynucleotide kinase-phosphatase (PNKP) mutations and neurologic disease. Mech. Ageing Dev. 161, 121-129 (2017).
Ferrand J., Rondinelli B., Polo S. E., Histone Variants: Guardians of Genome Integrity, Cells 2020 Nov. 5; 9 (11): 2424. doi.org/10.3390/cells9112424.
Freschauf G K et al, Identification of a small molecule inhibitor of the human DNA repair enzyme polynucleotide kinase/phosphatase, Cancer Res. 2009 Oct. 1; 69 (19): 7739-46
Hashizume, R. et al. Pharmacologic inhibition of histone demethylation as a therapy for pediatric brainstem glioma. Nat. Med. 20, 1394-1396 (2014).
Jilani A, Ramotar D., Slack C., Ong C., Yang X. M., Scherer S. W., Lasko D. D., Molecular cloning of the human gene, PNKP, encoding a polynucleotide kinase 3′-phosphatase and evidence for its role in repair of DNA strand breaks caused by oxidative damage J biol Chem 1999 Aug. 20; 274 (34): 24176-86
Kalasova I, Hanzlikova H, Gupta N, Li Y, Altmüller J, Reynolds JJ, Stewart GS, Wollnik B, Yigit G, Caldecott KW. Novel PNKP mutations causing defective DNA strand break repair and PARP1 hyperactivity in MCSZ Neurol Genet. 2019 Mar. 25; 5 (2): e320.
Karimi-Busheri F., Daly G., Robins P, Canas B, Pappin D. J., Sgouros J., Miller G. G. Fakrai H., Davis E. M.,
Le Beau M. M., Weinfeld M., Molecular characterization of a human DNA kinase, J biol Chem 1999 Aug. 20; 274 (34): 24187-94.
Lim, J. et al. Transcriptome and protein interaction profiling in cancer cells with mutations in histone H3.3. Sci. Data 5, 180283 (2018).
Lowe B. R. et al. Surprising phenotypic diversity of cancer-associated mutations of Gly 34 in the histone H3 tail. eLife 10, e65369 (2021).
Mereniuk T R et al, Genetic screening for synthetic lethal partners of polynucleotide kinase/phosphatase: potential for targeting SHP-1-depleted cancers Cancer Res. 2012 Nov. 15; 72 (22): 5934-44.
Mereniuk, T. R. et al. Synthetic lethal targeting of PTEN-deficient cancer cells using selective disruption of polynucleotide kinase/phosphatase. Mol. Cancer Ther. 12, 2135-2144 (2013).
Moras A. M., Henn J. G., Reinhardt L. S., Lenz G., Moura D. J., Recent developments in drug delivery strategies for targeting DNA damage response in glioblastoma, Life sciences 2021 Dec. 15; 287:120128.
Nagaraja, S. et al. Transcriptional Dependencies in Diffuse Intrinsic Pontine Glioma. Cancer Cell 31, 635-652.e6 (2017)
Pfister, S. X. et al. SETD2-Dependent Histone H3K36 Trimethylation Is Required for Homologous Recombination Repair and Genome Stability. Cell Rep. 7, 2006-2018 (2014).
Plessier, A., Dret, L. L., Varlet, P., Beccaria, K., Lacombe, J., Mériaux, S., Geffroy, F., Fiette, L., Flamant, P., Chrétien, F., et al. (2017). New in vivo avatars of diffuse intrinsic pontine gliomas (DIPG) from stereotactic biopsies performed at diagnosis. Oncotarget 8, 52543-52559. doi.org/10.18632/oncotarget.15002.
Ragazzini, R. et al. EZHIP constrains Polycomb Repressive Complex 2 activity in germ cells. Nat. Commun. 10, 3858 (2019).
Roux, K. J., Kim, D. I., Raida, M., and Burke, B. (2012). A promiscuous biotin ligase fusion protein identifies proximal and interacting proteins in mammalian cells. J. Cell Biol. 196, 801-810.
Scott, W. A. & Campos, E. I. Interactions With Histone H3 & Tools to Study Them. Front. Cell Dev. Biol. 8, 701 (2020).
Sirbu, B. M., Couch, F. B. & Cortez, D. Monitoring the spatiotemporal dynamics of proteins at replication forks and in assembled chromatin using isolation of proteins on nascent DNA. Nat. Protoc. 7, 594-605 (2012).
Söderberg, O. et al. Direct observation of individual endogenous protein complexes in situ by proximity ligation. Nat. Methods 3, 995-1000 (2006).
Soutoglou, E. & Misteli, T. Activation of the cellular DNA damage response in the absence of DNA lesions. Science 320, 1507-1510 (2008).
Weinberg, D. N., Allis, C. D. & Lu, C. Oncogenic Mechanisms of Histone H3 Mutations. Cold Spring Harb. Perspect. Med. 7, a026443-a026443 (2017).
Yadav, R. K. et al. Histone H3G34R mutation causes replication stress, homologous recombination defects and genomic instability in S. pombe. eLife 6, (2017).
Werbrouck, C., Evangelista, C. C. S., Lobón-Iglesias, M.-J., Barret, E., Teuff, G. L., Merlevede, J., Brusini, R., Berggruen, T., Mondini, M., Bolle, S., et al. (2019). TP53 Pathway Alterations Drive Radioresistance in Diffuse Intrinsic Pontine Gliomas (DIPG). Clin Cancer Res 25, 6788-6800. doi.org/10.1158/1078-0432.ccr-19-0126.

1. Inhibitor of the bifunctional polynucleotide kinase/phosphatase (PNKP) enzyme for use for inhibiting or preventing the proliferation of tumor cells bearing at least one H3 oncohistone mutation in a subject, said mutation inducing an increased binding of PNKP to mutated histone or to damaged replication forks in said tumor cells.

2. Inhibitor of PNKP for use according to claim 1, wherein said H3 oncohistone mutation affects histone variants H3.3 or H3.1.

3. Inhibitor of PNKP for use according to claim 1, wherein said H3 oncohistone mutation affects histone variants H3.3.

4. Inhibitor of PNKP for use according to claim 1, wherein said tumor cells have unmutated PTEN, ING3, CDKN3, PTPN6 and/or SMG1 genes and/or normal expression of PTEN, ING3, CDKN3, PTPN6 and/or SMG1.

5. Inhibitor of PNKP for use according to claim 1, wherein said tumor cells are chosen in the group consisting of: glioma, osteosarcoma, adrenocortical carcinoma, giant cell tumor of bone, chondroblastoma and acute myeloid leukemia (AML).

6. Inhibitor of PNKP for use according to claim 1, wherein said tumor cells are glioma cells bearing at least one H3.3 oncohistone mutation showing increased binding of PNKP to mutated histone or to damaged replication forks in said tumor cells.

7. Inhibitor of PNKP for use according to claim 1, wherein said tumor cells bear at least one mutation affecting the histone variant H3.3, said mutation being chosen in the group consisting of: G34W, K36M, K27M and G34R.

8. Inhibitor of PNKP for use according to claim 1, wherein said tumor cells are glioma cells, such as paediatric glioma cells, bearing the mutation K27M or G34R on histone variant H3.3.

9. Inhibitor of PNKP for use according to claim 1, wherein said tumor cells are giant cell tumor of bone cells bearing the mutation G34W in histone variant H3.3 or are chondroblastoma cells bearing the mutation K36M in histone variant H3.3.

10. Inhibitor of PNKP for use according to claim 1, wherein said inhibitor is a small chemical drug, a peptide, an antibody, an aptamer or an interferent nucleic acid.

11. Inhibitor of PNKP for use according to claim 1, wherein said inhibitor is a siRNA or a miRNA inhibiting the expression of the PNKP gene.

12. Inhibitor of PNKP for use according to claim 1, wherein said inhibitor is a siRNA whose sequence is disclosed in SEQ ID NO:3, SEQ ID NO:4 or SEQ ID NO: 26.

13. Inhibitor of PNKP for use according to claim 11, wherein said siRNA or miRNA is associated with magnetic nanoparticles, nanotubes, liposomes, polymeric nanoparticles, microvesicles, implants or micelles.

14. Inhibitor of PNKP for use according to claim 1, wherein said inhibitor is administered in combination with a chemotherapeutic or a radiotherapeutic treatment in a patient diagnosed which said tumor.

15. An in vitro use of an inhibitor of the bifunctional polynucleotide kinase/phosphatase (PNKP) enzyme for inhibiting or preventing the proliferation of tumor cells bearing at least one H3 oncohistone mutation, said mutation inducing an increased binding of PNKP to mutated histone or to damaged replication forks in said tumor cells.


SEQUENCE LISTING (26 sequences)

SEQ ID NO: 1 - DNA, 1731 bp, Homo sapiens
  misc_feature 1..1731: note = NM_007254.4 homo sapiens, mRNA of PNKP
  source 1..1731: mol_type = other DNA
atttccgtccgccgaggaaccgaccgccgccggccgggttgcaggcggggcacctcgggcaggacctccctggtcggaagtggccgtgagcccaagccgcggtcccgggccggcacccaggatgggcgaggtggaggccccgggccgcttgtggctcgagagcccccctgggggagcgccccccatcttcctgccctcggacgggcaagccctggtcctgggcaggggacccctgacccaggttacggaccggaagtgctccagaactcaagtggagctggtcgcagatcctgagacccggacagtggcagtgaaacagctgggagttaacccctcaactaccgggacccaggagttgaagccggggttggagggctctctgggggtgggggacacactgtatttggtcaatggcctccacccactgaccctgcgctgggaagagacccgcacaccagaatcccagccagatactccgcctggcacccctctggtgtcccaagatgagaagagagatgctgagctgccgaagaagcgtatgcggaagtcaaaccccggctgggagaacttggagaagttgctagtgttcaccgcagctggggtgaaaccccagggcaaggtggctggctttgatctggacgggacgctcatcaccacacgctctgggaaggtctttcccactggccccagtgactggaggatcttgtacccagagattccccgtaagctccgagagctggaagccgagggctacaagctggtgatcttcaccaaccagatgagcatcgggcgcgggaagctgccagccgaggagttcaaggccaaggtggaggctgtggtggagaagctgggggtccccttccaggtgctggtggccacgcacgcaggcttgtaccggaagccggtgacgggcatgtgggaccatctgcaggagcaggccaacgacggcacgcccatatccatcggggacagcatctttgtgggagacgcagccggacgcccggccaactgggccccggggcggaagaagaaagacttctcctgcgccgatcgcctgtttgccctcaaccttggcctgcccttcgccacgcctgaggagttctttctcaagtggccagcagccggcttcgagctcccagcctttgatccgaggactgtctcccgctcagggcctctctgcctccccgagtccagggccctcctgagcgccagcccggaggtggttgtcgcagtgggattccctggggccgggaagtccacctttctcaagaagcacctcgtgtcggccggatatgtccacgtgaacagggacacgctaggctcctggcagcgctgtgtgaccacgtgtgagacagccctgaagcaagggaaacgggtcgccatcgacaacacaaacccagacgccgcgagccgcgccaggtacgtccagtgtgcccgagccgcgggcgtcccctgccgctgcttcctcttcaccgccactctggagcaggcgcgccacaacaaccggtttcgagagatgacggactcctctcatatccccgtgtcagacatggtcatgtatggctacaggaagcagttcgaggccccaacgctggctgaaggcttctctgccatcctggagatcccgttccggctatgggtggagccgaggctggggcggctgtactgccagttctccgagggctgagccccgcccagctcccctccacaataaacgctgtttctccttga

SEQ ID NO: 2 - protein, 521 aa, Homo sapiens
  REGION 1..521: note = MISC_FEATURE - NP_009185.2, peptide sequence of PNKP human
MGEVEAPGRLWLESPPGGAPPIFLPSDGQALVLGRGPLTQVTDRKCSRTQVELVADPETRTVAVKQLGVNPSTTGTQELKPGLEGSLGVGDTLYLVNGLHPLTLRWEETRTPESQPDTPPGTPLVSQDEKRDAELPKKRMRKSNPGWENLEKLLVFTAAGVKPQGKVAGFDLDGTLITTRSGKVFPTGPSDWRILYPEIPRKLRELEAEGYKLVIFTNQMSIGRGKLPAEEFKAKVEAVVEKLGVPFQVLVATHAGLYRKPVTGMWDHLQEQANDGTPISIGDSIFVGDAAGRPANWAPGRKKKDFSCADRLFALNLGLPFATPEEFFLKWPAAGFELPAFDPRTVSRSGPLCLPESRALLSASPEVVVAVGFPGAGKSTFLKKHLVSAGYVHVNRDTLGSWQRCVTTCETALKQGKRVAIDNTNPDAASRARYVQCARAAGVPCRCFLFTATLEQARHNNRFREMTDSSHIPVSDMVMYGYRKQFEAPTLAEGFSAILEIPFRLWVEPRLGRLYCQFSEG

SEQ ID NO: 3 - RNA, 19 nt, synthetic construct
  misc_feature 1..19: note = siPNKP_3'UTR_1
  source 1..19: mol_type = other RNA
cagctcccctccacaataa

SEQ ID NO: 4 - RNA, 19 nt, synthetic construct
  misc_feature 1..19: note = siPNKP_3'UTR_2
  source 1..19: mol_type = other RNA
cctccacaataaacgctgt

SEQ ID NO: 5 - DNA, 25 bp, synthetic construct
  misc_feature 1..25: note = Primer upstream of the CMV promoter
  source 1..25: mol_type = other DNA
tggcagtacatctacgtattagtca

SEQ ID NO: 6 - DNA, 20 bp, synthetic construct
  misc_feature 1..20: note = Primer N-terminal to SNAP
  source 1..20: mol_type = other DNA
gctggtgaaagtaggcgttg

SEQ ID NO: 7 - DNA, 33 bp, synthetic construct
  misc_feature 1..33: note = mutagenesis primer sequence K27M forward
  source 1..33: mol_type = other DNA
caaaagccgctcgcatgagtgcgccctctactg

SEQ ID NO: 8 - DNA, 33 bp, synthetic construct
  misc_feature 1..33: note = mutagenesis primer sequence K27M reverse
  source 1..33: mol_type = other DNA
cagtagagggcgcactcatgcgagcggcttttg

SEQ ID NO: 9 - DNA, 34 bp, synthetic construct
  misc_feature 1..34: note = mutagenesis primer sequence G34R forward
  source 1..34: mol_type = other DNA
gcgccctctactggaagggtgaagaaacctcatc

SEQ ID NO: 10 - DNA, 34 bp, synthetic construct
  misc_feature 1..34: note = mutagenesis primer sequence G34R reverse
  source 1..34: mol_type = other DNA
gatgaggtttcttcacccttccagtagagggcgc

SEQ ID NO: 11 - DNA, 34 bp, synthetic construct
  misc_feature 1..34: note = mutagenesis primer sequence G34V forward
  source 1..34: mol_type = other DNA
gcgccctctactggagtggtgaagaaacctcatc

SEQ ID NO: 12 - DNA, 34 bp, synthetic construct
  misc_feature 1..34: note = mutagenesis primer sequence G34V reverse
  source 1..34: mol_type = other DNA
gatgaggtttcttcaccactccagtagagggcgc

SEQ ID NO: 13 - DNA, 34 bp, synthetic construct
  misc_feature 1..34: note = mutagenesis primer sequence G34W forward
  source 1..34: mol_type = other DNA
gcgccctctactggatgggtgaagaaacctcatc

SEQ ID NO: 14 - DNA, 34 bp, synthetic construct
  misc_feature 1..34: note = mutagenesis primer sequence G34W reverse
  source 1..34: mol_type = other DNA
gatgaggtttcttcacccatccagtagagggcgc

SEQ ID NO: 15 - DNA, 32 bp, synthetic construct
  misc_feature 1..32: note = mutagenesis primer sequence K36M forward
  source 1..32: mol_type = other DNA
ctaccggcggggtgatgaagcctcatcgctac

SEQ ID NO: 16 - DNA, 32 bp, synthetic construct
  misc_feature 1..32: note = mutagenesis primer sequence K36M reverse
  source 1..32: mol_type = other DNA
gtagcgatgaggcttcatcaccccgccggtag

SEQ ID NO: 17 - DNA, 25 bp, synthetic construct
  misc_feature 1..25: note = siRNA siFANCD2
  source 1..25: mol_type = other DNA
ccatgtctgctaaagagcgttcatt

SEQ ID NO: 18 - DNA, 19 bp, synthetic construct
  misc_feature 1..19: note = siRNA siH3.3 (H3F3A)
  source 1..19: mol_type = other DNA
ctacaaaagccgctcgcaa

SEQ ID NO: 19 - DNA, 20 bp, synthetic construct
  misc_feature 1..20: note = siRNA siH3.3 (H3F3B)
  source 1..20: mol_type = other DNA
gctaagagagtcaccatcat

SEQ ID NO: 20 - DNA, 19 bp, synthetic construct
  misc_feature 1..19: note = siRNA siLUC (luciferase control)
  source 1..19: mol_type = other DNA
cgtacgcggaatacttcga

SEQ ID NO: 21 - DNA, 19 bp, synthetic construct
  misc_feature 1..19: note = siRNA siPNKP_1
  source 1..19: mol_type = other DNA
ccggatatgtccacgtgaa

SEQ ID NO: 22 - DNA, 19 bp, synthetic construct
  misc_feature 1..19: note = siRNA siRNF168
  source 1..19: mol_type = other DNA
ggcgaagagcgatggagga

SEQ ID NO: 23 - DNA, 19 bp, synthetic construct
  misc_feature 1..19: note = siRNA siSETD2
  source 1..19: mol_type = other DNA
gtgaaggagtatgcacgaa

SEQ ID NO: 24 - DNA, 19 bp, synthetic construct
  misc_feature 1..19: note = siRNA siPNKP_2
  source 1..19: mol_type = other DNA
ggaaacgggtcgccatcga

SEQ ID NO: 25 - DNA, 19 bp, synthetic construct
  source 1..19: mol_type = other DNA
  misc_feature 1..19: note = siRNA siXRCC4
atatgttggtgaactgaga

SEQ ID NO: 26 - RNA, 19 nt, synthetic construct
  source 1..19: mol_type = other RNA
  misc_feature 1..19: note = siPNKP_3'UTR_3
ccacaataaacgctgtttc